(19) United States
(12) Patent Application Publication
(10) Pub. No.: US 2023/0039091 A1
(43) Pub. Date: Feb. 9, 2023

(54) METHODS AND SYSTEMS FOR NON-INVASIVE FORECASTING, DETECTION AND MONITORING OF VIRAL INFECTIONS

(71) Applicant: Empatica Srl, Milano (IT)

(72) Inventors: Simone TOGNETTI, Vimodrone (IT); Giulia REGALIA, Milano (IT); Andrea MORTERA, Turin (IT); Matteo LAI, Boston, MA (US); Rosalind PICARD, Newtonville, MA (US); Francesco ONORATI, Cambridge, MA (US)

(21) Appl. No.: 17/967,293

(22) Filed: Oct. 17, 2022

Related U.S. Application Data
(63) Continuation of application No. PCT/US2021/027988, filed on Apr. 19, 2021.
(60) Provisional application No. 63/011,833, filed on Apr. 17, 2020.

(51) Int. Cl. A61B 5/00; A61B 5/0205; A61B 5/11; A61B 5/0531; G16H 50/80; G16H 50/30

(52) U.S. Cl. CPC A61B 5/418; A61B 5/02055; A61B 5/7275; A61B 5/7267; A61B 5/1112; A61B 5/6801; A61B 5/0531; A61B 5/7278; G16H 50/80; G16H 50/30; A61B 2560/0247; A61B 5/024

(57) ABSTRACT

Devices, systems, and methods herein relate to non-invasive patient monitoring for infection detection and infection resolution. These systems and methods may receive and measure patient biosignals to estimate an infection level of a patient. In some embodiments, a method may include the steps of receiving physiological data of a patient. An infection measure may be estimated based on the physiological data. An infection state of the patient may be detected based at least in part on the estimated infection measure.

### CROSS-REFERENCE TO RELATED APPLICATIONS

This application claims the benefit of U.S. Provisional Application No. 63/011,833, filed Apr. 17, 2020, the content of which is hereby incorporated by reference in its entirety.

### TECHNICAL FIELD

Devices, systems, and methods herein relate to non-invasive user monitoring to detect a viral infection.

### BACKGROUND

Vital signs such as temperature, heart rate, and blood pressure are commonly used to indicate the status and health of a user. For example, a patient who suspects that they may have contracted a virus may aid diagnosis of their condition by taking a temperature measurement. Active infection monitoring is typically performed after a patient expresses symptoms (e.g., discomfort, pain, fever, fatigue, cough). However, some patients may be infected with a virus such as COVID-19 or influenza but may be asymptomatic for a period of time (e.g., a number of days) after the initial onset of the infection. Furthermore, infected patients may be unsure which virus they contracted, and thus may be unsure of a therapy to seek. These patients may go on to unwittingly infect others they come into contact with including their partners, family, friends, co-workers, and community. Therefore, additional methods, device, and systems for detecting and identifying viral infection may be desirable.

### SUMMARY

Described here are systems, devices, and methods for non-invasively monitoring user infection. The human immune system response may be regulated in part by the sympathetic nervous system (SNS). For example, the first immediate response of inflammation may be regulated by the SNS where central sympathetic activity may have a direct impact on inflammatory cytokines. Lymphoid tissue may be highly innervated by sympathetic nerve fibers as well, with sympathetic nerve terminals located close to immune cells. Generally, the early onset of infection may be detected by continuously measuring the SNS such as by, for example, measuring changes in electrodermal activity which involves measuring not only a “sweat response” but also sensitive changes in electrical properties that occur in the skin even in the absence of a “sweat response” that looks or feels like sweat on the surface of the skin. In some embodiments, a method of detecting infection of a patient may include receiving physiological data of a patient, measuring electrodermal activity of the patient using a non-invasive patient measurement device, estimating a measure associated with a probability or likelihood of infection based at least in part on the combination of heart rate data, temperature data, and the electrodermal activity measurement and/or physiological data (e.g., probability estimate for viral infection or an estimate of viral concentration), and detecting an infection state of the patient based at least in part on the estimated viral concentration. In one embodiment, the user device determines patient-specific historical baseline metrics for a predetermined period of time. As physiological parameters are measured, the user device identifies measurements deviating from the baseline metrics. In response to a predetermined deviation, such as a predetermined combination of heart rate, temperature and electrodermal activity deviations, a notification of infection is provided.

In some embodiments, a method may include receiving, from one or more sensors, physiological data of a patient measured by the one or more sensors, the physiological data including movement data associated with the patient, identifying a resting time period during which the patient is at rest based on the movement data, extracting, from the physiological data, data of a set of physiological parameters during a portion of the resting time period, determining a derived measure for each physiological parameter from the set of physiological parameters based on the data of the set of physiological parameters extracted during the portion of the resting time period, adjusting the derived measure for each physiological parameter from the set of physiological parameters based on a baseline measure for the corresponding physiological parameter from the set of physiological parameters to produce a set of adjusted measures, the baseline measure associated with physiological data of the patient collected during a time period prior to the resting time period, inputting the set of adjusted measures into a model for predicting an infection risk of the patient to obtain a predictive value indicative of an infection level of the patient, and determining an infection state of the patient based on the predictive value.

In some embodiments, the derived measure for each physiological parameter from the set of physiological parameters is an aggregate value of the data extracted for that physiological parameter during the portion of the resting time period. In some embodiments, the aggregate value is based on one or more of: a mean, a median, a standard deviation, a variance, or a higher order statistic.

In some embodiments, the set of physiological parameters includes two or more of: heart rate, heart rate variability, skin temperature, respiration rate, skin conductance, skin resistance, skin potential, motion, blood-oxygen level, protein, or cytokines.

In some embodiments, the adjusting the derived measure for each physiological parameter from the set of physiological parameters includes determining a change between the derived measure and the baseline measure for each physiological parameter from the set of physiological parameters.

In some embodiments, the method may include receiving at least one of demographic data or medical data of the patient, and adjusting the data of the set of physiological parameters extracted during the portion of the resting time period based on the at least one of the demographic data or the medical data, the determining the derived measure for each physiological parameter from the set of physiological parameters being after the normalizing the rest data. In some embodiments, the method may include adjusting the model for predicting an infection risk of the patient based on at least one of the demographic data or the medical data.

In some embodiments, the method may include normalizing the data of the set of physiological parameters extracted during the portion of the resting time period. In some embodiments, determining the derived measure for each physiological parameter from the set of physiological parameters is after the normalizing the rest data. In some embodiments, normalizing the data is based on minimum-maximum normalization.

In some embodiments, determining the infection state of the patient includes: determining whether the predictive value is greater than a predefined threshold value, and in response to the predictive value being greater than the predefined threshold value, determining that the patient is infected.

In some embodiments, the predefined threshold value may be adjustable. In some embodiments, the model may be calibrated using training data including physiological data associated with a set of users, the set of users and the patient having a common set of characteristics.

In some embodiments, the model may be configured to apply a set of weights to the set of adjusted measures, the set of weights being calibrated using training data including physiological data associated with a set of users. In some embodiments, the physiological data of the set of users may be associated with one or more of a geolocation and weather.

In some embodiments, the model defines a non-linear function. In some embodiments, the time period prior to the resting time period is at least about 24 hours prior to the resting time period. In some embodiments, the time period prior to the resting time period is a predefined period of time prior to the resting time period, the predefined period of time based on a type of infection.

In some embodiments, the method may include monitoring the infection state of the patient over time to identify one or more of a change in the infection state of the patient, an infection resolution, and estimating a duration of an infection.

In some embodiments, the time period and the resting time period are based on a periodic time interval. In some embodiments, the periodic time interval comprises one or more of a calendar cycle, hormonal cycle, lunar cycle, circadian rhythm, and multidien rhythm, and work schedule.

In some embodiments, adjusting the derived measure is based on a weather associated with the patient. In some embodiments, the method may include determining an infection risk based at least in part on a geolocation of the patient.

In some embodiments, an apparatus may include a memory, and a processor operatively coupled to the memory and a set of sensors, the processor configured to execute instructions stored in the memory to: receive, from the set of sensors, physiological data of a patient measured by the set of sensors, extract, from the physiological data, data of a set of physiological parameters during a resting time period associated with the patient, determine a derived measure for each physiological parameter from the set of physiological parameters based on the data of the set of physiological parameters extracted during the resting time period, adjust the derived measure for each physiological parameter from the set of physiological parameters based on a baseline measure for the corresponding physiological parameter from the set of physiological parameters to produce a set of adjusted measures, the baseline measure associated with physiological data of the patient collected during a time period prior to the resting time period, determine a predictive value indicative of an infection level of the patient using a model for predicting an infection risk of the patient and the set of adjusted measures, and determine an infection state of the patient based on the predictive value.

In some embodiments, that apparatus may include a wearable device including the set of sensors. In some embodiments, the derived measure for each physiological parameter from the set of physiological parameters is an aggregate value of the data extracted for that physiological parameter during the portion of the resting time period. In some embodiments, the set of physiological parameters includes two or more of: heart rate, heart rate variability, skin temperature, respiration rate, skin conductance, blood-oxygen levels, skin resistance, skin potential, motion, or cytokines. In some embodiments, the processor may be configured to execute the instruction to adjust the derived measure for each physiological parameter from the set of physiological parameters by determining a change between the derived measure and the baseline measure for each physiological parameter from the set of physiological parameters.

In some embodiments, the model may be configured to apply a set of weights to the set of adjusted measures, the set of weights being calibrated using training data including physiological data associated with a set of users. In some embodiments, the model may define a non-linear function.

### BRIEF DESCRIPTION OF THE DRAWINGS

FIG. 1 is a block diagram of a system for detecting user infection, according to an embodiment.

FIG. 2 is a flow chart illustrating a method of detecting user infection, according to an embodiment.

FIG. 3 is a flow chart illustrating a method of notifying user infection, according to an embodiment.

FIGS. 4A-4B are plots of user infection data from an example study, according to an embodiment. FIG. 4A is a plot of viral shedding over time, according to an embodiment. FIG. 4B is a plot of symptoms over time, according to an embodiment.

FIGS. 5A-5E are plots of physiological data from an example study, according to an embodiment. FIG. 5A is a plot of acceleration over time, according to an embodiment. FIG. 5B is a plot of skin conductance over time, according to an embodiment. FIG. 5C is a plot of temperature over time, according to an embodiment. FIG. 5D is a plot of heart rate over time, according to an embodiment. FIG. 5E is a linear combination of the plots depicted in FIGS. 5A-5D, according to an embodiment.

FIGS. 6A-6B are plots of physiological data from an example study, according to an embodiment. FIG. 6A is a plot of viral shedding over time, according to an embodiment. FIG. 6B is a plot of symptom counts over time, according to an embodiment.

FIGS. 7A-7E are plots of physiological data from an example study, according to an embodiment. FIG. 7A is a plot of acceleration over time, according to an embodiment. FIG. 7B is a plot of skin conductance over time, according to an embodiment. FIG. 7C is a plot of temperature over time, according to an embodiment. FIG. 7D is a plot of heart rate over time, according to an embodiment. FIG. 7E is a linear combination of the plots depicted in FIGS. 7A-7D, according to an embodiment.

FIGS. 8A-8C are plots of physiological data of a patient infected with SARS-CoV-2, according to an embodiment. FIG. 8A is a plot of heart rate over time, according to an embodiment. FIG. 8B is a plot of temperature over time, according to an embodiment. FIG. 8C is a plot of electrodermal activity, according to an embodiment.

FIG. 9 depicts a resting time period of a user, according to embodiments.

### DETAILED DESCRIPTION

Described here are infection monitoring devices, systems, and methods for providing real-time, non-invasive monitoring of one or more physiological parameters, which may be used to detect viral infection of a user (e.g., patient). The methods, systems, and devices disclosed herein may be particularly useful in early (e.g., asymptomatic) respiratory infections such as COVID-19 and influenza. These systems and methods may, for example, receive physiological data of a patient (e.g., heart rate, temperature, acceleration, cytokine content in sweat), time-of-day information, and environmental data (e.g., geolocation, weather, air quality), measure electrodermal activity (EDA) (e.g., skin conductance) of the patient, and estimate a probability or likelihood of viral infection (or some measure associated therewith) and responses that tend to accompany infection such as the SNS response associated with cytokine release or with a so-called cytokine storm based at least on the EDA and physiological data, and detect an infection state based on the estimated probability. This may, for example, allow early insight into a patient's health status on a continuous or semi-continuous, real-time basis. Furthermore, infection resolution of a patient may be detected based on a change in physiological data or infection states of a patient so as to facilitate monitoring of a patient's health status (e.g., whether a patient's condition is improving, decreasing, or remaining the same). The devices described herein for use in detecting infection may be compact and portable such that they may allow for continuous or semi-continuous, real-time monitoring without restricting the day-to-day activities of a patient. For example, the device may be a wearable device worn on a patient's wrist.

Also described herein are infection monitoring devices, systems, and methods for providing real-time notification of infection detection and/or infection resolution. For example, infection resolution parameters may include a time of infection resolution. In some embodiments, an alert may be provided to one or more of a patient, designated user, and health care professional upon detection of a viral infection. One or more notification parameters such as frequency, timing, type, severity, and content may be configurable for each patient. For example, a notification may indicate a confidence level (e.g., low, medium, high, probability value, risk level) that the patient has an infection of a specific type of virus.

The methods and system described herein may be a first line of defense for patients and health care professionals for continuous and non-invasive infection monitoring. Early detection and notification may allow, for example, infected patients to be prioritized for further testing (e.g., a PCR/lab test to confirm infection (and determine the type of infection)) while the patient is still asymptomatic. In this manner, viral infection may be forecasted in that symptoms associated with viral infection may be predicted before being exhibited or felt by a patient. Early detection and notification of infection may also allow treatment to begin while patients are asymptomatic or patient symptoms are mild. This may reduce the duration and severity of illness and thereby improve patient outcomes. Once notified of possible infection, the patient may choose to self-isolate before symptoms appear and thus reduce the ability of the virus to spread. A patient's condition may also be continuously monitored while the patient is infected to determine the efficacy of a patient's treatment (e.g., is the patient improving?). For example, if a large surge in EDA is measured together with one or more of more rapid respiration, higher heart-rate, and higher temperature, combined with ongoing low physical activity level, then a notification may be generated to one or more of the patient and health care provider for further intervention due to a possible cytokine storm. IL-6, IL-12, other protein levels may be checked for possible changes, as certain patterns in these physiological markers may be associated with an increased likelihood of more severe symptoms and/or health complications.

Additionally or alternatively, health care professionals may remotely monitor patients using the systems, devices, and methods described herein on a more frequent basis than intermittent clinic visits, thereby reducing potential exposure of health care providers to highly contagious patients. Current standard of care often relies on a combination of patient visual appearance, blood tests (e.g., serologic test), and vital measurements. The data provided from the monitoring system can supplement the traditional data sets and provide additional insights into patient status, and account for a patient's historical (e.g., reference baseline) status. Continuous (or periodic) patient monitoring also allows health care professionals to address complications and/or poor treatment efficacy in real-time before issues exacerbate. For example, when treatment efficacy is poor, the prescribed medical therapy (e.g., drug, dosage, frequency) can be updated without an in-person consultation. Early detection of infection combined with early treatment and intervention steps (e.g., self-isolation) may reduce the R₀ (e.g., R₀<1) of viruses such as COVID-19 and influenza to reduce the impact of such diseases on patients, society, and the economy.

In some embodiments, measured physiological data may be used to increase understanding of viruses. For example, patient data measured over a period of time for one or more patients may allow for data trending visualization. For example, measured physiological data and behavioral data may be used to determine why two patients with the same viral load and/or identical genes may have vastly different symptom severity. For example, a patient who is less physically active and obtains more sleep, even with the same fever and same viral load, might shorten or lengthen their number of days of symptoms and contagiousness. Such data may be further useful in clinical trials for detecting inflammatory events that may create variance in patient outcomes.

### I. SYSTEMS

Generally, the systems described here may include a user device (e.g., patient monitor including one or more devices with sensors) and one or more other compute devices, e.g., a partner device, a network, a server, and a database. The user device may measure patient data, and may, in some embodiments, transmit the patient data to another compute device, partner device, remote server, and/or database for processing and analysis. In other embodiments, the patient data may be processed and analyzed by the user device itself. As mentioned above, the patient data may include one or more of physiological data, environmental data, and demographic data. The physiological data may be measured using one or more sensors of one or more devices (e.g., wearable device, smartphone, portable device). The patient data may be processed and analyzed to estimate a probability of likelihood of infection (or some measure associated therewith) and/or detect an infection state of a patient. The measurement of patient data and infection detection may be performed for predetermined intervals or continuously. The results of the infection detection may be output to one or more of the patient monitor, compute device, partner device, network, server, database, combinations thereof, and the like. Additionally (e.g., concurrently or subsequently) or alternatively, the infection detection may be output to one or more of a health care professional and designated users (e.g., partner, family, support group, researchers).

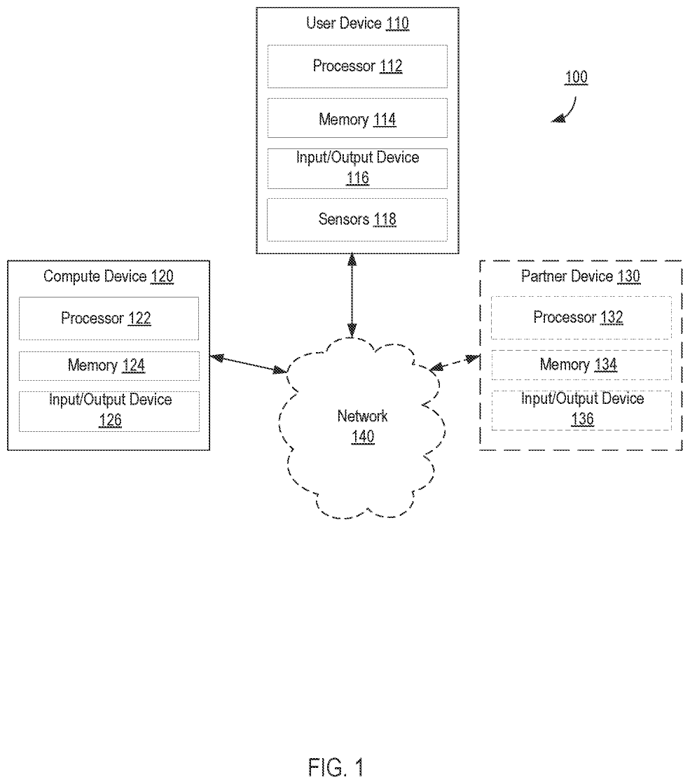

A patient monitoring system may include one or more of the components necessary to measure and/or generate physiological data using the devices as described herein. FIG. 1 is a block diagram of an embodiment of a patient monitoring system (100). As shown there, the system (100) may include a user device (e.g., patient monitor) (110), a compute device (120), and a network (140), and optionally a partner device (130).

In some embodiments, measured patient data may be processed and/or analyzed (e.g., for an indication of infection) on any one of the devices of the system (100) (e.g., user device (110), compute device (120)), while in other embodiments, the processing may be distributed throughout a plurality of devices. In some embodiments, patient data processing may include filtering data (e.g., reducing noise, averaging waveforms), determining key events (e.g., wake period, sleep period, periods of time with low, moderate, or high physical activity or outdoor air exposure), estimating viral infection, detecting patient infection, and monitoring infection changes (e.g., whether a patient's condition is changing, decreasing, increasing, or staying the same), infection resolution, and estimating a duration of an infection. In some embodiments, patient data and patient identifying information may be encrypted and stored according to Health Insurance Portability and Accountability Act (HIPAA) regulations.

The user device (110) can be a compute device that is associated with a user. The user device (110) can be configured to removably attach to a patient and measure patient data (e.g., physiological data, environmental data). As described in more detail herein, the user device (110) may include one or more of an electrodermal activity sensor, accelerometer, cardiac sensor, optical sensor, temperature sensor, magnetometer, altimeter, 1-lead electrocardiogram (ECG) sensor, electromyography (EMG) sensor, or ambient light sensor. In some embodiments, the user device (110) may be configured to be a wearable device, e.g., worn on a patient's limb (e.g., wrist, arm, calf).

The user device (110) may further include a communication device (e.g., as part of input/output device (116)) configured to establish a communication channel with one or more other devices or systems. For example, the user device (110) may be coupled to compute devices through one or more wired or wireless communication channels. The user device (110) may be operatively coupled one or more compute devices (120), partner devices (130), and/or networks (140), as further detailed below.

The user device (110) may be configured to measure patient data (e.g., physiological data, electrodermal activity data) during a plurality of time periods. In some embodiments, the measured patient data may be transmitted to a compute device for data processing, infection probability estimation and forecasting, and infection detection as described herein. In some embodiments, the user device (110) may be controlled from one or more other compute devices. In some embodiments, the user device (110) described herein may be configured to perform a subset of the measurement, estimation, and detection steps described herein.

In some embodiments, a user device (110) (e.g., patient monitor) may include a processor (112), memory (114), input/output device (116), and one or more sensors (118). The processor, memory, and input/output device are described in more detail herein. The one or more sensors (118) may be configured to measure one or more physiological parameters and environmental parameters. In some embodiments, the physiological parameters may include patient activity (e.g., movement), patient position, sleep/wake status, sleep quality, NREM/REM staging, electrodermal activity (e.g., skin conductance), blood volume, heart rate, heart rate index, heart rate variability, blood pressure, respiration, respiration rate index, metabolic equivalent of task (MET), quantity and type of motion (MOT), stress level, relaxation level, temperature, skin temperature, skin resistance, skin potential, motion, skin conductance, blood-oxygen level, heat flux, ANS activity (e.g., indicating levels of sympathetic, parasympathetic, and enteric nervous system arousal or activation), proteins (e.g., cytokines, Interleukin-6 (IL-6) and Interleukin-12 (IL-12), corresponding physiologically relevant indexes, combinations thereof, and the like.

In some embodiments, one or more parameters may be derived from a set of other parameters. For example, heart rate and respiration may be obtained through analysis of changes in seismocardiography motion parameters that change with the motion generated by the beating heart or breathing lungs. In some embodiments, heart rate variability (HRV) may be measured through changes in those motion parameters, or through the skin, or in the blood beneath the skin of the patient. HRV may be defined as the beat-to-beat variations in heart rate. The larger the alterations, the larger the HRV. HRV includes two primary components: respiratory sinus arrhythmia (RSA) which is also referred to as high frequency (HF) oscillations, and low frequency (LF) oscillations. HF oscillations are associated with respiration and track the respiratory rate across a range of frequencies, and low frequency oscillations are associated with Mayer waves (Traube-Hering-Mayer waves) of blood pressure. The total energy contained by these spectral bands in combination with the way energy is allocated to them gives an indication of the heart rate regulation pattern given by the central nervous system, and an indication of the state of mental and physical health.

In some embodiments, the environmental parameters may include geolocation, weather, air pressure, temperature, humidity, pollen count, air quality, local infection rate, population density, combinations thereof, and the like.

In some embodiments, the sensor (118) may include an electrodermal activity sensor, accelerometer, gyroscope, photoplethysmography sensor, cardiac sensor (e.g., electrocardiography), blood oxygen sensor, optical sensor, geolocation sensor (e.g., GPS), barometer, pressure sensor, temperature sensor (e.g., skin temperature sensor), glucose sensor, barometer, electrodes, AC current sensor, DC current sensor, light emitter, magnetometer, altimeter, 1-lead electrocardiogram (ECG) sensor, electromyography (EMG) sensor, ambient light sensor, cytokine sensor, protein sensor, combinations thereof, and the like. In some embodiments, the accelerometer may include one or more of a 3-axis accelerometer and gyroscope configured to measure one or more of movement and position. For example, the accelerometer or gyroscope may be configured to quantify the duration a patient is lying down. In some embodiments, the total daily duration that the patient is not lying down can be monitored as a metric for patient activity. A predetermined deviation from the reference baseline duration may be configured to output a notification (e.g., alert, instruction).

The processor (112) of the user device (110) may incorporate data received from memory (114) of the user device (110) and over a communication channel to control one or more components, e.g., of the user device (110). The memory (114) may further store instructions to cause the processor (112) to execute modules, processes and/or functions associated with the methods described herein. In some embodiments, the memory (114) and processor (112) may be implemented on a single chip. In other embodiments, they can be implemented on separate chips.

In some embodiments, the user device (110) may be coupled directly to any of the compute device (120), partner device (130), and network (140). The network might also provide wireless (e.g. WiFi) processing of the physiological data of the user, which may be combined with data from the wearable. For example, WiFi may be used to estimate respiration and heart-rate, which might be combined with EDA, temperature, or IL-6 from the wearable. In some embodiments, the user device (110) may be coupled to a dock (not shown) for storage, to recharge, to transfer data, combinations thereof, and the like. The user device (110) may include a power source configured to provide electrical power to the user device (110) and/or the user device (110) can be coupleable to an external power source (e.g., via the dock). In some embodiments, the wearable device can include a wrist band or other attachment mechanism (e.g. magnet, adhesive, etc.) for attaching to a user.

Suitable examples of devices for measuring physiological parameters of a user include, for example, devices such as those described in U.S. Patent Application Publication No. 2014/0316229, titled “Apparatus for electrodermal activity measurement with current compensation,” filed Mar. 17, 2014, U.S. Patent Application Publication No. 2015/0327787, titled “Device, system and method for detection and processing of heartbeat signals,” filed Jul. 24, 2015, and U.S. Pat. No. 8,140,143, filed Apr. 16, 2009, titled “Washable wearable biosensor,” the contents of each of which are incorporated herein by reference.

The compute device (120) may be a remote device that is operatively coupled to or integrated with the user device (110) and/or a partner device (130), e.g., via network (140). In some embodiments, the compute device (120) can be associated with a server. The server can be a dedicated server that receives data and signals from and sends data and signals to one or more user device(s) (110). Alternatively, the compute device (120) can be another user device. For example, the compute device (120) can be a cellular telephone (e.g., smartphone), tablet computer, laptop computer, desktop computer, portable media player, etc.

The compute device (120) may be configured to receive various types of data. For example, the compute device may be configured to receive demographic data (e.g., gender, weight, Body Mass Index, height, birthday, age, height, genetic information, diagnosis date, anniversary date using the device, pre-existing conditions, medication history, medical history), patient data (e.g., blood pressure data, heart rate data, electrodermal activity data), general health information of other similarly situated patients (e.g., cohort patient data), or any other relevant information. For example, medical history may include a history of one or more of respiratory illness, immune-system disease, hypertension, cardiovascular disease, diabetes, and the like.

In some embodiments, the compute device (120) may be configured to create, receive, and/or store patient profiles. A patient profile may contain any of the patient demographic data previously described. While the above-mentioned information may be received by the compute device, in some embodiments, the compute device may be configured to process any of the above data from information it has received using software stored on the device itself, or externally.

The processor (122) of the compute device (120) can be configured to receive patient data from the user device (110) and other data (e.g., environmental data, demographic data) from other sources (e.g., partner device (130)). The processor (122) may be configured to receive, process, analyze, compile, store, and access data. The processor (122) may be configured to receive data directly input and/or measured from a patient. The processor (122) may receive the data through a network connection, as discussed in more detail herein, or through a physical connection with the device or storage medium (e.g. through Universal Serial Bus (USB) or any other type of port).

The partner device (130) may be a compute device that is associated with a healthcare facility, research facility, etc. In some embodiments, an individual such as a health care professional may be allowed access to the user device (110) through a partner device (130).

The network (140) can be any type of network (e.g., a local area network (LAN), a wide area network (WAN), a virtual network, a telecommunications network) implemented as a wired network and/or wireless network and used to operatively couple compute devices, including user devices (110), compute devices (120), and/or partner devices (130). The communication may or may not be encrypted. A wireless network may refer to any type of digital network that is not connected by cables of any kind. Examples of wireless communication in a wireless network include, but are not limited to cellular, radio, satellite, and microwave communication. However, a wireless network may connect to a wired network in order to interface with the Internet, other carrier voice and data networks, business networks, and personal networks. A wired network is typically carried over copper twisted pair, coaxial cable and/or fiber optic cables. There are many different types of wired networks including wide area networks (WAN), metropolitan area networks (MAN), local area networks (LAN), Internet area networks (IAN), campus area networks (CAN), global area networks (GAN), like the Internet, and virtual private networks (VPN). The network (140) may include or be coupled to one or more databases and servers for processing and/or storage.

Processors (112, 122, 132) may be any suitable processing device configured to run and/or execute a set of instructions or code and may include one or more data processors, image processors, graphics processing units, physics processing units, digital signal processors, and/or central processing units. Each processor (112, 122, 132) may be, for example, a general purpose processor, Field Programmable Gate Array (FPGA), an Application Specific Integrated Circuit (ASIC), and/or the like. Each processor (112, 122, 132) may be configured to run and/or execute application processes and/or other modules, processes and/or functions associated with the system and/or a network associated therewith. The underlying device technologies may be provided in a variety of component types (e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and/or the like.

Each memory (114, 124, 134) may include a database (not shown) and may be, for example, a random access memory (RAM), a memory buffer, a hard drive, an erasable programmable read-only memory (EPROM), an electrically erasable read-only memory (EEPROM), a read-only memory (ROM), Flash memory, and the like. Each memory (114, 124, 134) may store instructions to cause the processor to execute modules, processes, and/or functions associated with the communication device, such as patient data processing, sensor measurement, viral infection probability estimation, user device or patient monitoring control, authentication, encryption, and/or communication. Some embodiments described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for the specific purpose or purposes.

Examples of non-transitory computer-readable media include, but are not limited to, magnetic storage media such as hard disks, floppy disks, and magnetic tape; optical storage media such as Compact Disc/Digital Video Discs (CD/DVDs); Compact Disc-Read Only Memories (CD-ROMs), and holographic devices; magneto-optical storage media such as optical disks; solid state storage devices such as a solid state drive (SSD) and a solid state hybrid drive (SSHD); carrier wave signal processing modules; and hardware devices that are specially configured to store and execute program code, such as Application-Specific Integrated Circuits (ASICs), Programmable Logic Devices (PLDs), Read-Only Memory (ROM), and Random-Access Memory (RAM) devices. Other embodiments described herein relate to a computer program product, which may include, for example, the instructions and/or computer code disclosed herein.

Each input/output device (116, 126, 136) is configured to permit a patient, health care professional, or other user to control one or more of the devices of the system. For example, an input/output device (126) of the compute device (120) may include an input device for a user to input commands and an output device for a user to receive output (e.g., electrodermal activity readings on a display device). Each input/output device (116, 126, 136) may include a network interface configured to connect the compute device to another system (e.g., Internet, remote server, database) by wired or wireless connection (e.g., via network (140)). In some embodiments, the network interface may include a radiofrequency (RF) receiver, transmitter, and/or optical (e.g., infrared) receiver and transmitter configured to communicate with one or more devices and/or networks. The network interface may communicate by wires and/or wirelessly with one or more of the user device (110), compute device (120), partner device (130), and network (140).

In some embodiments, an output device of a compute device (e.g., incorporated in an input/output device (116, 126, 136)) may output viral infection probability estimates (e.g., infection risk), metrics associated with a current state or physiological condition of a user, etc. and may include one or more of a display device and audio device. Data analysis may be displayed by the output device (e.g., display). Data used in infection detection such as physiological data and patient data may be and output visually and/or audibly through one or more output devices. In some embodiments, an output device may include a display device including at least one of a light emitting diode (LED), liquid crystal display (LCD), electroluminescent display (ELD), plasma display panel (PDP), thin film transistor (TFT), organic light emitting diodes (OLED), electronic paper/e-ink display, laser display, and/or holographic display. In some embodiments, the output device can include an audio device. The audio device may audibly output patient data, physiological data, system data, alarms and/or notifications. For example, the audio device may output an audible alarm when viral infection probability reaches a predetermined infection threshold or when a malfunction in the user device (110) is detected. In some embodiments, an audio device may include at least one of a speaker, piezoelectric audio device, magnetostrictive speaker, and/or digital speaker. In some embodiments, a patient may communicate with other users using the audio device and a communication channel. For example, a patient may form an audio communication channel (e.g., VoIP call) with a remote health care professional.

In some embodiments, an input device of a compute device (e.g., input/output device (116, 126, 136)) may include at least one switch configured to generate a control signal. For example, an input device may include a touch surface for a user to provide input (e.g., finger contact to the touch surface) corresponding to a control signal. An input device comprising a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies. In embodiments of an input device comprising at least one switch, a switch may include, for example, at least one of a button (e.g., hard key, soft key), touch surface, keyboard, analog stick (e.g., joystick), directional pad, mouse, trackball, jog dial, step switch, rocker switch, pointer device (e.g., stylus), motion sensor, image sensor, and microphone. A motion sensor may receive patient movement data from an optical sensor and classify a user gesture as a control signal. A microphone may receive audio data and recognize a patient voice as a control signal.

In some embodiments, a haptic device may be incorporated into an input/output device (e.g., input/output device (116)) to provide additional sensory output (e.g., force feedback) to the user. For example, a haptic device may generate a tactile response (e.g., vibration) to confirm user input to an input device (e.g., touch surface). As another example, haptic feedback may notify that user input is overridden by the compute device. In some embodiments, a haptic device can be used to send alerts to a user (e.g., when a user has a detected infection).

### II. METHODS

Also described here are methods for non-invasively monitoring infection of a patient using the systems and devices described herein (e.g., user device (110), compute device (120), etc.). In particular, the systems, devices, and methods described herein may be used to estimate a measure such as viral infection probability to detect patient infection in real-time and/or to estimate a measure indicative of infection changes (e.g., improvements or lack thereof over time). For example, the patient monitors described herein enable non-medical professionals (e.g., patients) to continuously or semi-continuously, non-invasively, and discretely determine and track their infection level without interrupting their daily activities. The devices, systems, and methods, may be easy for a patient to use and require little training, allow for a private infection detection and monitoring while in public, and provide a comfortable and portable way to determine and track health status. Moreover, the patient monitors may remain comfortably, continuously wearable for several days to several weeks without interfering in a patient's activities and without significant up-keep.

In some embodiments, a set of physiological data and environmental data may establish a reference baseline which may be used to detect patient infection. A patient's reference baseline may correspond to a steady state (e.g., healthy) condition and/or non-infected state of the patient. In some embodiments, the reference baseline may include patient activity, patient position, sleep/wake status, sleep quality, NREM/REM staging, electrodermal activity, blood volume, heart rate, heart rate variability, blood pressure, respiration, metabolic equivalent of task (MET), stress level, relaxation level, patient temperature (e.g., skin temperature), heat flux, ANS activity, proteins, geolocation, weather, air pressure, temperature (e.g., environmental), humidity, pollen count, air quality, local infection rate, population density, combinations thereof, and the like. In some embodiments, motion data (e.g., patient activity) may be used to determine a time period of the reference baseline. For example, motion data of the patient may be used to determine non-sleep data of the patient that may be excluded from further processing and analysis. That is, heart rate data, temperature data, and electrodermal activity data of an awake patient (e.g., in motion) may not be useful for infection detection and resolution.

In some embodiments, a user device such as user device (110) can be configured to measure physiological data of a patient and provide that physiological data to a processor (e.g., processor (112), processor (122)) for further processing and/or analysis. In some embodiments, the user device can be configured to measure data associated with a plurality of physiological parameters. In some embodiments, the physiological parameters can include one or more of heart rate indexes (e.g., measures computed from heart rate), respiration rate indexes (e.g., measures computed from respiration rate), heart rate variability indexes (e.g., time-domain, frequency-domain, complexity indexes or other measures computed from heart rate or respiration rate), skin temperature indexes (e.g., measures computed from skin temperature or any processed/cleaned skin temperature derived data), electrodermal activity indexes (e.g., measures computed from electrodermal activity or processed/cleaned electrodermal activity derived data), or quantity and type of motion indexes (e.g., measures computed from accelerometer data, gyroscope data, electromyography data, or other source of information related to motion). In some embodiments, the physiological parameters can include a set of two, three, four, or five different physiological parameters. The processor can be configured to determine, based on the physiological data, a baseline value for each of the physiological parameters. In some embodiments, the processor can be configured to analyze the data to determine whether a patient is at rest, and in response to determining that the patient is at rest, determine a baseline value for each physiological parameter at rest (e.g., during a resting time period).

In some embodiments, a notification may be provided to one or more users (e.g., patient, health care professional) when infection is detected. In some embodiments, one or more notifications (e.g., alerts) may be provided to a predetermined set of contacts (e.g., family, partner, caregiver, health care professional) based on predetermined criteria. The set of contacts may be selected, for example, by the patient or a caregiver, and may receive notifications or other communications via one or more of a telephone call, email, text message, push notification on a mobile device, web portal, and the like. In some embodiments, the notification may include one or more of patient infection status (and/or patient infection resolution status), infection confidence level, infection type, a behavioral (e.g., health) recommendation, and a prompt (e.g., request) for patient input. For example, a prompt for patient input may include one or more questions to reduce false positive infections (e.g., “Have you taken any new medications or substances in the last 24 hours that might have affected your overall physiology?”). In some embodiments, the patient input in response to the prompt may be incorporated into a model (e.g., machine learning model) as described in more detail herein.

In some embodiments, one or more of frequency, timing, and content of patient notification may be modified based on patient data such as demographic data. For example, patients who are in a high-risk group (e.g., elderly, pre-existing condition, socioeconomic status) may receive a patient infection notification based on a lower estimated viral infection probability than a patient in a low-risk group (e.g., young, healthy).

Additionally or alternatively, in some embodiments, the infection state of a patient may be utilized to remotely monitor and/or manage a patient. For example, patients with known risk factors (e.g., diabetes, hypertension, obesity, age) may be more actively and comprehensively monitored using mobile applications and third-party interaction. For example, health care professionals (e.g., primary care physicians) may use the information to adjust a medication regimen, clinical assessments, and/or to inform therapy decision-making. In some embodiments, a health care professional (e.g., care provider) may be provided access to the patient data via a graphical user interface on one or more compute devices such as through a browser-based web access portal. The health care professional may, for instance, log in to the browser-based web access portal via a personal computer. The health care professional could review all of the monitored data from one or more patients. The health care professional could additionally input lab test results, notes from patient appointments, patient therapy changes, patient infections and complications, and any other findings into the patient's health record. In some embodiments, patient infection data may be transmitted to an authorized organization (e.g., health organization, university, research group). For example, patient data may be anonymized to remove identifiable features and transmitted to the Centers for Disease Control and Prevention for tracking. Anonymous patient infection data may be incorporated into viral infection models to forecast viral infections prior to conventional self-reporting and testing methods.

In some embodiments, patient infection data of a group of patients may also lead to better evidence-based treatment recommendations over time. In some embodiments, machine learning techniques may be utilized to analyze large sets of patient infection data to determine and refine the physiological, demographic, and environmental parameters that correspond to one or more of infection, treatment outcomes, and risk. For example, changes in electrodermal activity at rest relative to a set of reference heart rate, temperature, and electrodermal activity at rest may be highly correlated with patient infection due to COVID-19 and other viruses such as influenza. As another example, data trends may show that reducing stress and increasing sleep duration or quality during or preceding the onset of patient infection may reduce the duration and severity of illness.

### Methods for Detecting Infection

The methods for detecting infection may generally include receiving patient data (e.g., demographic data, medical data) and environmental data, measuring physiological data (e.g., electrodermal activity, heart rate variability) of the patient using a non-invasive patient measurement device, estimating a viral infection probability (or other measure of infection) based at least in part on the physiological data measurement and other data, and detecting an infection state of the patient based at least in part on the estimated viral infection probability. In some embodiments, the first physiological data may include a reference set of baseline data of a first time period and the second physiological data may be of a second time period. In some embodiments, the viral infection probability of the patient may be estimated based on a change in the patient data (e.g., physiological data, demographic data, environmental data, medical data) between the first and second time periods. In some embodiments, the viral infection probability estimate may be further based on a machine learning algorithm or regression model (e.g., non-linear model) that receives as input a reference physiological data of a patient and cohort patient data associated with the patient. It should be appreciated that any of systems and devices described herein may be used in the methods described here.

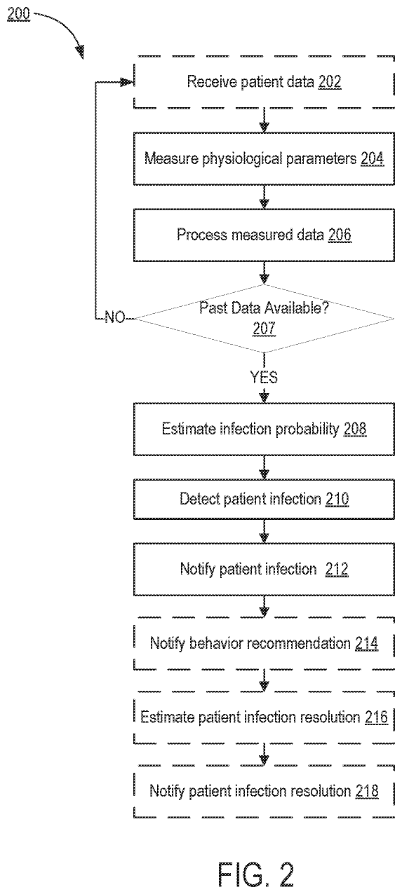

FIG. 2 is a flowchart depicting an illustrative embodiment of a method of detecting infection (200). In the embodiment depicted in FIG. 2, the method may include receiving patient data (202), measuring physiological parameters (204), process measured data (206), estimate viral infection probability (208), detect patient infection (210), notify patient infection (212), and notify behavior recommendation (214).

Optionally, in step 202, patient data may be received at a device (e.g., user device (110), compute device (120)). For example, the patient data may include time-stamped reference data including one or more of physiological data (e.g., symptoms data), demographic data, and environmental data. The reference patient data may be received once, or at predetermined intervals (e.g., weekly, biweekly, monthly), and may be based on data from one or more devices including the user device, compute device, partner device, network, and the like. For example, the patient may input their demographic information into the user device (e.g., smartphone) or partner device (doctor's tablet). In some embodiments, the reference patient data may correspond to a first time period such as a healthy and/or non-infected state. In some embodiments, the received patient data may be stored in memory of a user device (110) and/or compute device (120). In some embodiments, the patient data may be received from a compute device (e.g., partner device (130)) associated with a healthcare facility, a weather facility, a government agency, a marketing agency, etc. In some embodiments, the patient data may be received using a wireless communication channel. In some embodiments, the reference data may include measurements taken under different physiological conditions such as a resting condition (e.g., sleeping) and non-resting conditions such as during various levels of physical and mental activity.

In some embodiments, the received patient data may include cohort physiological data associated with the patient. For example, the patient may be classified into a cohort (e.g., demographic group, peer group), which may include a set of patients grouped by one or more of age, gender, race, and body mass index, and/or grouped by behavior characteristics (e.g. high or low sleep regularity). For example, the patient may input demographic data into a compute device including information such as their age, gender, race, weight, height, body mass index, and the like to determine the cohort of a patient. Also, user device data measured over time maybe used to characterize behaviors. In some embodiments, the cohort physiological data may be pre-programmed and stored in memory of the user device (110) or may be received (e.g., updated) over a communication channel. In some embodiments, the cohort physiological data may be processed using machine learning techniques.

In some embodiments, patient data may be measured and/or derived continuously. For example, patient sleep and/or rest data (e.g., sleep or rest start/end times, duration, sleep efficiency, sleep fragmentation, number of turns-and-tosses) may be estimated from sensor and/or physiological data and/or physiological indexes. In some embodiments, patient data may include device usage data such as one/off wrist data corresponding to when the user device (110) is worn correctly by the patient when the user device (110) is a wrist worn device.

In step 204, one or more physiological parameters may be measured using a user device. For example, a user device (e.g., user device (110)) may use one or more sensors (e.g., sensors (118)) on a continuous or semi-continuous, real-time basis. In some embodiments, a patient monitor may measure one or more physiological and environmental parameters of a patient. For example, a patient monitor may be releasably attached flat to the skin of the patient over the wrist. The sensors of the patient monitor may be configured to measure physiological parameters such as electrodermal activity and environmental parameters such as location, continuously or at pre-determined intervals while the patient performs any of their daily activities. Analyzing a plurality of physiological parameters may improve infection detection by reducing false positive and false negatives due to factors such as sensor mis-calibration (e.g., loose device on patient wrist) or other conditions such as coughing or exercise. In some embodiments, measuring physiological parameters may include performing calibration of the user device including one or more sensors.

In some embodiments, physiological parameters may be measured during a first time period and a second time period after the first time period. Additionally or alternatively, one or more demographic and environmental parameters may be measured in the same time period (e.g., first time period, second time period) as the physiological measurements. The set of measured data during the first time period may be used to determine a baseline value for one or more physiological parameters, as described herein. The set of measured data during the second time period may be referred to as measured patient data. The measured parameters may be used to determine patient infection during the second time period. In some embodiments, the second time period may be between about 5 seconds and about one week, including all sub-values in-between, after the first time period. For example, the second time period may be up to about 1 minute, up to about 5 minutes, up to about 10 minutes, up to about 30 minutes, up to about 60 minutes, up to about 2 hours, up to about 6 hours, up to about 12 hours, up to about 24 hours, up to about 36 hours, up to about 48 hours, up to about 72 hours, up to about 96 hours, up to about 120 hours, up to about 1 week, after the first time period.

In some embodiments, the baseline value may change over time and/or be based on periodic time intervals/periods, as more patient data is measured. For example, the first and second time periods may be periodic (e.g., Monday night sleep, first week of month, yearly date, season, hormonal cycle, menstrual cycle, lunar cycle, circadian rhythm, multidien rhythm, work schedule, school schedule) and/or set based on patient input. For example, a time period and a resting time period may be based on a periodic time interval. The periodic time interval may comprise one or more of a calendar cycle, hormonal cycle, lunar cycle, circadian rhythm, and multidien rhythm, and work schedule

In some embodiments, patient activity may include acceleration measurements using a patient monitor including a 3-axis accelerometer. In some embodiments, blood volume pulse (BVP) may be measured using a photoplethysmography (PPG) sensor on a user device (110). In some embodiments, heart rate, temperature, and electrodermal activity may be measured using a set of sensors, for example, one or more of the devices as described in the patent publications attached as Attachment A and B. The sensor may measure one or more of skin conductance, skin impedance, skin potential, and skin resistance. The sensor may be capable of measuring skin conductance, skin impedance, skin potential, and/or skin resistance at a sufficient resolution for determining a measure of infection.

In some embodiments, patient geolocation may be measured over time using, for example, GPS and/or WiFi. Geolocation data may be used to determine movement patterns such as sleep location, commute patterns, and the like. Deviations from regular habits may indicate heightened stress and increased susceptibility to infection. In some embodiments, patient geolocation may be used to determine environmental parameters such as weather, air quality, pollen, and the like. Weather and environmental parameters may impact respiratory function, mood, and stress, one or more of which may impact susceptibility to a viral infection. In some embodiments, calendar information (e.g., from calendaring software) can be associated with geolocation data, e.g., to better understand a location of a patient and their interactions. In some variations, physiological data of a set of users may be associated with one or more of a geolocation and weather. In some variations, a derived measure may be adjusted based on a weather associated with the patient.

In some embodiments, proteins may be measured using the user device and/or separate protein measurement device. For example, proteins such as IL-6, IL-12, and other interleukins modified by an early cytokine response to a viral inflammation may indicate viral infection.

In some embodiments, sleep parameters may be derived from other measurements such as acceleration, EDA, BVP, and temperature. Sleep parameters may include main sleep period onset time, offset time, interruptions, turns and tosses, effective rest duration, efficiency, fragmentation, awakenings per first three hours of the main sleep period, duration of awakenings, nap periods and their duration over a 24 hour period.

In some embodiments, temperature may be measured using a temperature sensor of a user device. Temperature parameters may include mean, median, and standard deviation, frequency (e.g., 11 bins) of normalized skin temperature for day time, entire sleep and 1-4 quarters of sleep. In some embodiments, temperature measurements may be analyzed in context of daily activities (e.g., rest, exercise, walking).

In step 206, the measured patient data may be processed. For example, the raw measured patient data may be processed (e.g., to remove or clean certain data such as outliers by filtering, averaging, etc.) and produce processed patient data that may be further analyzed. In some embodiments, the measured patient data may be processed to classify the measured patient data by the patient's sleep/wake cycle (e.g., wake state, sleep/wake transition state, sleep state). In some embodiments, a patient's sleep/wake cycle may be determined based on, for example, time, acceleration, position, temperature, and heart rate data. Further analysis and processing of the measured physiological parameters may be based on the sleep/wake cycle. For example, viral infection probability estimates during a wake state may be based on temperature, electrodermal activity, acceleration, and heart rate. Viral infection probability estimates during a sleep/wake state may be based on sleep duration, fragmentation, and sleep storm. Viral infection probability estimates during a sleep state may be based on temperature, electrodermal activity, heart rate, and heart rate variability. In some embodiments, infection detection, monitoring, or resolution estimation during a sleep state may be based on indexes or measures associated with (e.g., derived from) body or skin temperature, electrodermal activity (e.g., skin conductance), and heart rate. In some embodiments, infection detection, monitoring, or resolution estimation during a sleep state may be based on indexes or measures derived from heart rate, body or skin temperature, electrodermal activity, respiratory rate, motion, or any combination thereof.

In some embodiments, physiological parameters, such as heart rate and heart rate quality features (minimum rates, maximum variability), may be analyzed differently during sleep and wake periods. For example, illness may raise the resting HR, lower the HRV HF component and the PPSD, raise the LF/HF ratio, and raise the EDA's skin conductance level. These patterns of change may be compared to personalized baseline patterns, taken relative to a similar time of day of a day having similar activity patterns.

In some embodiments, processing of acceleration measurements may include mean, median, standard deviation of the 3-axis RMS values, and frequency analysis of the RMS values for the separate rest and physical activities. In some embodiments, frequency analysis of acceleration data may provide information about heart rate and respiration. In some embodiments, an identity of a user of the user device (110) may be determined based on one or more of measured acceleration data and blood volume pulse (BPV). In some embodiments, the user device may restrict use when an identity of a user of the user device cannot be confirmed based on measured acceleration and BPV data.

In some embodiments, patient activity may be measured throughout a day as a function of acceleration and be classified with activity levels (e.g., activity signatures) including sleep, awake rest/low activity, medium activity or intense activity, walk and run activities.

In some embodiments, acceleration measurements may be used in processing other physiological parameters. For example, movement data (e.g., acceleration measurements) may indicate that certain time periods may be susceptible to motion-induced artifacts and error. As another example, skin conductance levels may be higher for a predetermined amount of time after physical activity, as it is associated with an increase in overall arousal, and not only with exercise-induced sweating.

In some embodiments, BVP data may be interpolated (e.g., over-sampled) and used to refine peak positions to derive mean peak-to-peak (PP) intervals (e.g., heart rate estimation) and heart rate variability (HRV) measures including low-frequency (LF) and high-frequency (HF) components, ratio of LF/HF, standard deviation of PP intervals, and changes in envelope parameters that carry respiration data. In some embodiments, cardiac parameter values derived from BVP measurements may be processed based on one or more of acceleration, electrodermal activity, and temperature in order to compensate for changes in heart rate due to stress rather than physical activity. In some embodiments, BVP data may be processed to generate a BP quality score to determine a quality of the derived HR and HRV values. BVP data having a low BP quality score may be excluded from further data analysis such as infection detection.

In some embodiments, electrodermal activity measurements may be processed to generate one or more of physiological parameters including mean EDA, median EDA, gradient, peak count per time interval, and normalized skin conductance level (SCL) captured during sleep, wake, and different daily routine or atypical activities. In some embodiments, the SCL may be normalized by the activity counts (e.g., derived from acceleration) and temperature. SCL may also be processed based on a sleep/wake cycle (e.g., daytime sitting, walking, different intervals of sleep). In some embodiments, the frequency and length of EDA storms during different intervals of sleep may also be calculated. In some embodiments, physiological parameters including one or more of sleep stages, autonomic stress levels, changes in medication, and physical activity may be based on EDA.

In some embodiments, sleep parameter values may be derived from previous nights (e.g., up to about month). Sleep parameter values may be processed to determine a regularity of sleep timing over predetermined periods (e.g., one week, two weeks).

Patient data during sleep or rest periods can be less affected by external factors unrelated to a physiological state or condition of a patient. Accordingly, in some embodiments, systems, devices, and methods can be configured to determine whether a patient is at rest (e.g., sleeping) and use the physiological data collected during sleep to monitor a physiological state of a patient. For example, systems and devices described herein (e.g., user device (110), compute device (120)) may be configured to apply an algorithm for detecting rest (e.g., an algorithm that evaluates acceleration over time, movement over time, etc.) to determine when a patient is at rest. Such systems and devices can, upon determining that a user is at rest, collect physiological data associated with that user during the rest period. In some embodiments, patient data (e.g., heart rate, temperature, electrodermal activity) may be processed for a sleep period where physiological indexes are present for at least about 1 hour, 2 hours, 3 hours, 4 hours, 5 hours, including any values or ranges therebetween. For example, in some embodiments, systems and devices described herein can analyze motion (e.g., accelerometer data) to determine a starting point and an ending point of a rest period for a user, and then collect physiological data on that user during that rest period. For rest periods having a duration less than a predefined threshold (e.g., about a few hours or less), systems and devices described herein can determine that such data is insufficient for evaluating infection of the user. For rest periods having a duration equal to or greater than the predefined threshold, systems and devices described herein can process the physiological data collected during those periods for infection detection. In some embodiments, a patient may be notified of insufficient data for infection prediction, monitoring, and/or resolution.

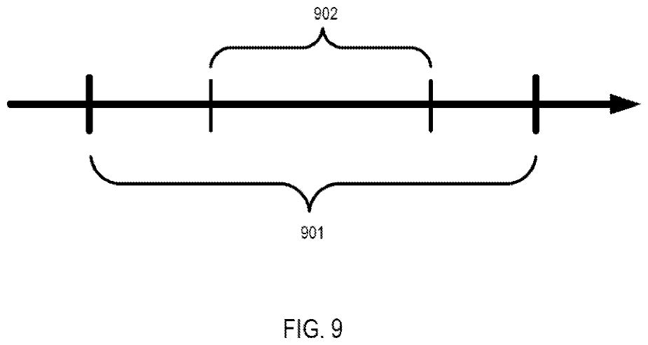

In some embodiments, when systems and devices described herein determine that a patient has a rest period (e.g., resting time period) that is sufficiently long (e.g., greater than a predefined threshold), such systems and devices may be configured to use the data from a central time period during (e.g., a portion of) the rest period to evaluate the patient for infection. The data from this central time period can less affected by external factors unrelated to a patient's physiological condition and therefore provide more reliable data for predicting infection and/or patient monitoring. For example, for a patient having an eight hour sleep period, the middle four hour period of sleep time (e.g., hours 2-6) may be used to derive the patient data used for patient infection detection and resolution as it may generally have less noise. In some embodiments, systems and devices described herein can be configured to extract the physiological data of the patient during a predefined time period (902) (e.g., about 2 hours, about 3 hours, about 4 hours, about 5 hours) falling in a middle of a patient's rest period (901) (e.g., a portion of the resting time period), as schematically depicted in FIG. 9. Such systems and devices can then use this information for infection detection, monitoring, or resolution prediction.

In some embodiments, current parameter values may be compared relative to baseline parameter values when predicting infection. At 207, systems and devices described herein can determine whether past data (e.g., baseline parameter values) is available, and if available, proceed to 208. The baseline values can be determined at an earlier period based on data collected during that earlier period. For example, the baseline values can be determined based on physiological data collected during a predefined period of time before current values. The predefined period separating the baseline value determination and the current measured value determination can be set to different values based on type of infection (e.g., influenza, COVID-19, or another type of virus or disease), demographic of a user, etc. The predefined time period may be about 3 days, about 4 days, or about 5 days for COVID-19 and about 1 day, about 2 days, or about 3 days for influenza or other types of cold viruses, since each type of virus or bacterial infection may have different physiological incubation periods before symptoms or other physiological changes may manifest themselves. In some embodiments, the predefined time period may be greater than about 24 hours, greater than about 36 hours, greater than about 48 hours, including all values and subranges in-between. Certain demographics, e.g., children, may generally have different incubation periods than other demographics, e.g., adults. The physiological incubation period may correspond to the time period between infection and detectable physiological changes in patient data. In some embodiments, the physiological incubation period may be determined from past assessments of a particular disease (e.g., by physicians and/or using machine learning techniques). In some embodiments, the physiological incubation period can be less than a symptomatic incubation period. A symptomatic incubation period may correspond to the time period between infection and patient detectable symptomatic changes in patient data. The symptomatic incubation period may be greater than the physiological incubation period as physiological changes can occur before the patient exhibits symptoms of infection.

In some embodiments, physiological data derived or extracted from one or more sleep periods (e.g., resting time periods) and/or wake periods may be processed to generate a set of baseline values for those parameters. For example, systems, devices, and methods described herein can determine a derived measure for each physiological parameter. In some embodiments, derived measures can include one or more aggregate measures (e.g., mean, median, standard deviation, variance, higher order statistics and/or their combination and/or transformation) that are computed on the physiological data collected for each parameter (e.g., raw and/or processing physiological data). In some embodiments, a minimum-maximum normalization scheme can be employed to normalize the baseline values and/or physiological data. In some embodiments, demographic data or other physiological data may be used to adjust (e.g., normalize) the baseline values and/or physiological data for different subgroups (e.g., demographic groups, medical data). For example, one or more of the skin conductance and heart rate may be normalized by one or more of temperature and activity level.

In step 208, viral infection probability (or some other measure associated with such probability, e.g., an indication of viral concentration) may be estimated, e.g., by the user device (e.g., user device (110)) or a remote compute device (e.g., compute device (120) associated with a server accessible via a network). For example, the measured physiological parameters of a current time period (e.g., second time period) may be compared against the reference patient data of a past time period (e.g., first time period) to estimate a viral infection probability. The viral infection probability estimates may be used to monitor a patient infection state. For example, deviation of the measured physiological parameters from the reference patient data may indicate an increase in viral infection probability (e.g., viral load estimate).

In some embodiments, current measured values for one or more parameters can be adjusted or normalized based on past values (e.g., baseline values, baseline measure) for those parameters and/or using minimum-maximum normalization. For example, a current measured value for that parameter can be adjusted based on a past value (e.g., baseline value, baseline measure) for that parameter. In some embodiments, an aggregate value of the current measured data for each parameter from a plurality of parameters can be determined, and a change (e.g., difference) between the aggregate value and an aggregate value corresponding to past measured data for each parameter from the plurality of parameters can be determined as adjusted measures. For example, a first aggregate value based on data collected for skin conductance during a first period of time can be determined, and a second aggregate value based on data collected for skin conductance during a second period of time after the first period of time can be determined, and a change (e.g., difference) between the first and second aggregate values can be determined to evaluate a risk of infection. Aggregate values can be any one of a mean, a median, a standard deviation, a variance, a higher order statistic, and/or a combination or transformation of any such measures. The adjusted measurement may be based on a predetermined time interval such as a periodic time interval (e.g., Monday night sleep, first week of month, yearly date, season, hormonal cycle, menstrual cycle, lunar cycle, circadian rhythm, multidien rhythm, work schedule, school schedule).

In some embodiments, values representing differences between current aggregate measures and past aggregate measures (e.g., baseline measures) for a set of physiological parameters can be determined, and such values can be used in combination to evaluate a risk of infection. For example, such values (e.g., adjusted measures) can be provided as inputs into a function (e.g., a machine learning model) that provides an output that represents a risk of infection of a user (e.g., for predicting an infection risk of the patient).

In some embodiments, each of the parameters may be provided a predetermined weight, which may be determined using one or more machine learning techniques described herein. In some embodiments, comparisons between reference data and measured data may be relative to one or more of a time period of the day, sleep/wake cycle, patient baseline, cohort baseline, combinations thereof, and the like. In some embodiments, thresholds and weightings of the parameters may be derived from machine learning techniques.

In some embodiments, a linear or non-linear algorithm (e.g., a machine learning model) can be used to output a value that represents a current condition or state of a user. In such embodiments, a probabilistic output may be computed as a linear or nonlinear function of the weighted and/or normalized aggregate parameter values (e.g., a weighted value representing a difference between a current aggregate value for a parameter and a past aggregate value (baseline value) for a parameter). For example, the algorithm can be a function with weighted values for different parameters that are determined based on one or more machine learning algorithms such as linear regression, k-nearest neighbors, logistic regression, linear or nonlinear support vector machine, decision tree, random forest, neural networks, combinations thereof, and the like. In some embodiments, the output may be normalized (e.g., scaled) and compared to a predetermined threshold to determine a risk level (e.g., high risk, low risk).

In some embodiments, systems and devices described herein can use a non-linear function to provide an output that represents a risk of infection of a user. The non-linear function can receive as inputs aggregate values for a plurality of parameters that have been adjusted based on baseline aggregate values for that plurality of parameters. In an embodiment, the adjusted aggregate value for each parameter can be determined using a baseline subtraction equation, where the aggregate value v_{baseline subtracted}=aggregate(v_{d})−aggregate(v_{d-2}, v_{d-3}, v_{d-4}), where aggregate(v_{d}) is the aggregate value of the parameter computed on day d, and aggregate(v_{d-2}, v_{d-3}, v_{d-4}) can be the aggregate value of the parameter from any one of days d−1, d−2, and d−3 or an aggregate of the aggregate values of the parameter from those days. For example, v_{baseline subtracted} for a parameter such as skin conductance can be a change (e.g., difference) between an aggregate measure of the physiological data collected on day d and an aggregate measure of the physiological data collected on day d−4. For different parameters, a different aggregate value may be used (e.g., a difference between day d and day d−1 may be sufficient for temperature while such may not be sufficient for skin conductance). For different infection types, a different aggregate value may be used (e.g., a difference between day d and day d−3 may be sufficient for an influenza infection while it may not be sufficient for a COVID-19 infection).

In some embodiments, where data for a particular day is not available (e.g., day d−4), systems and devices described herein can use data for another data (e.g., day d−3). Alternatively, if data extending back to a particular day is not available (e.g., no data is available before day d−3) and the predefined period required between a baseline measure and a current measure is insufficient (e.g., day d−3 is not a sufficient period of time from current day d), then systems and devices can indicate that insufficient data is available for determining risk of infection and/or monitoring infection/recovery progress. In some embodiments, missing data may be interpolated using multivariate models such as an autoencoder, and the uncertainty in the interpolated values may be carried forward into an uncertainty of subsequent decisions. While a subtraction between aggregate values associated with a parameter are described herein, it can be appreciated that any measure representative or indicative of a difference between current and past aggregate values associated with a parameter can be used, including, for example, a percentage difference, a deviation measure (e.g., a standard deviation), or more complex non-linear computations applied to compare multivariate dynamic patterns, etc.

In some embodiments, the non-linear function can apply different weights to the set of physiological parameters, where the weights were previously determined using training data. The training data can be associated with a particular infection type, and can be used to optimize or calibrate the function for determining infection risk based on the inputs of the patient aggregate values. In some embodiments, as further described with reference to 210 below, the output of the non-linear function can be compared to a threshold value. For example, if the output of the non-linear function is greater than or less than the threshold value, then systems and devices described herein can output an alert indicating that the user is infected.

In some embodiments, a viral infection probability estimate (e.g., infection risk) may be based at least in part on a geolocation of the patient. For example, a patient located in a high risk area (e.g., infection hotspot, high population density, public area) may have a higher risk of infection than a patient located in a low risk area. This may increase the probability estimate for viral infection for future days. This forecast might also inform recommendations and suggestions that the system provides (e.g., notifies) to the user, if the user configures the system to provide advice or behavior recommendations.

In step 210, patient infection may be detected, e.g., by the user device (e.g., user device (110)) or a remote compute device (e.g., compute device (120) associated with a server accessible via a network). In some embodiments, patient infection may be based on a predetermined threshold variable, e.g., determined on a per patient basis. For example, patients in a high risk cohort may have a lower viral infection probability threshold than patients in a low risk cohort. In some embodiments, as further described herein, a user can select the predetermined threshold variable based on their preferences or situation. For example, a user can select a lower or higher threshold value to adjust the sensitivity of the detection system (e.g., adjust whether the system provides more alerts with a higher false positive rate), such that a user who is high risk or close to someone who is high risk can tune the detection algorithm to be more sensitive and another user who is low risk and/or living alone can tune the detection algorithm to be less sensitive.

In step 212, patient infection may be notified. In some embodiments, a user device (e.g., user device (110)) may output one or more of a visual, audio, and haptic notification (e.g., alert) corresponding to positive patient infection. As described in more detail herein, patient notification settings may be based on a patient profile. The notification may be updated at periodic intervals when infection is detected. For example, the patient may be notified upon onset of infection and a change in rate of infection. In some embodiments, a patient may be prompted to provide input to an input device of the user device in response to receiving the notification. For example, the patient may be prompted to acknowledge the infection notification and/or input a rating of their subjective wellness. In some embodiments, a non-patient user such as a health care professional may be notified of the patient infection.

In some embodiments, a notification may be output when the measured patient data deviates from the baseline patient data by a predetermined threshold. In some embodiments, a notification sensitivity may be based on a number of deviations of measured physiological data from baseline patient data. That is, patients who are infected more often and for longer periods of time may receive notifications more frequently. In some embodiments, notification sensitivity may be set by the patient. In some embodiments, the notification may include a graphical representation of likelihood of infection compared to past infection events.

In some embodiments, a communication channel may be established between the patient and a health care professional in response to the notification of patient infection being sent to the patient. In some embodiments, one or more of a health care professional, a patient's partner, family member, and support group may receive one or more patient infection notifications. This may allow caregivers and stakeholders to become aware of infection at an early stage to allow early intervention with a patient when needed. Furthermore, additional notifications may be sent if the patient's condition worsen beyond a predetermined threshold.

Optionally, in step 214, a behavior recommendation may be notified. For example, a patient having a high likelihood of infection and who has recently had short sleep periods relative to their baseline sleep periods may receive a notification that lack of sleep may contribute to the severity of their infection and may encourage the patient to sleep or reduce stress more to reduce symptoms. In some embodiments, a behavior recommendation may be based on the patient's geolocation and medical history. For example, a patient with pre-existing conditions in a high risk zip code may receive a recommendation to relocate to another location. Behavior recommendations may be tailored to each patient and may include input from health care professionals. In some embodiments, the notification may also include a confidence level of the notification. For example, the notification may output a message such as, “There is a 30% chance you have a viral infection. Please continue to wear your device.”

Optionally, in step 216, patient infection resolution may be estimated by comparing patient infection values at different time periods to each other and/or a predetermined threshold. For example, a decreasing viral infection probability may correspond to infection resolution, while an increasing viral infection probability may correspond to lack of infection resolution.

Optionally, in step 218, patient infection resolution may be notified. In some embodiments, a user device (e.g., user device (110)) may output one or more of a visual, audio, and haptic notification (e.g., alert) corresponding to patient infection resolution. The notification may be updated at periodic intervals if a trend in the patient infection resolution changes. For example, the patient may be notified upon a decrease in the patient's viral infection probability. In some embodiments, a patient may be prompted to provide input to an input device of the user device in response to receiving the notification. For example, the patient may be prompted to acknowledge the infection resolution notification and/or input a rating of their subjective wellness. In some embodiments, a non-patient user such as a health care professional may be notified of the patient infection resolution (or lack thereof).

### Method of Notifying Patient Infection

In some embodiments, at least one notification may be outputted including one or more of the infection state of the patient, behavior recommendation, and patient data analysis. The notification may include notifying at least one predetermined contact including one or more of the patient, health care professional, patient's partner, caregiver, family member, and provider. A communication channel may be established between the patient and a health care professional in response to the notification corresponding to the patient being in a high-risk condition. A patient infection notification may be output based on one or more of the physiological parameter measurements such as electrodermal activity.

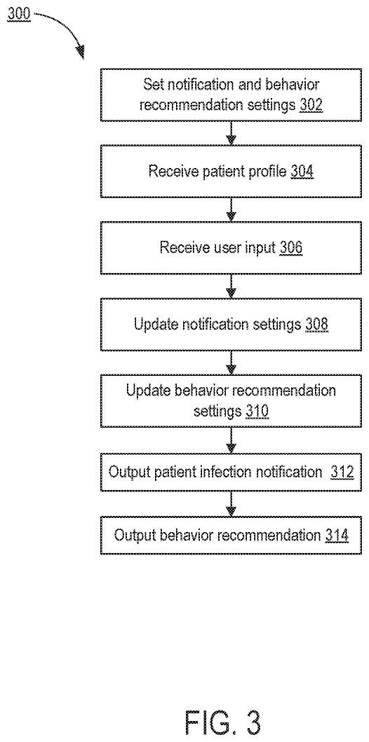

FIG. 3 is a flowchart depicting an illustrative embodiment of a method of notifying a patient and providing a behavior recommendation (300). In the embodiment depicted in FIG. 3, the method may include setting notification and behavior recommendation settings (302), receiving a patient profile (304), receiving user input (306), updating notification settings (308), updating behavior recommendation settings (310), outputting a patient infection notification (312), and outputting a behavior recommendation notification (314).

In step 302, notification and behavior recommendation settings may be set. Settings may include frequency, content, and type of notifications. Notification settings may also include communication method (e.g., text, SMS, e-mail, phone call, audio, graphic), language, complexity, detail level, and the like. In step 304, a patient profile may be received. A patient profile may contain any of the patient demographic data previously described. In some embodiments, older patients or patient in a high-risk cohort may receive more frequent notifications. In step 306, user input may be received. For example, a patient and/or health care professional may adjust the frequency, content, and timing of notifications based on their personal preferences. In some embodiments, a health care professional may update the behavior recommendations for the patient. In step 308, notification settings may be updated based on one or more of the patient profile and user input. In step 310, behavior recommendation settings may be updated based on one or more of the patient profile and user input. In step 312, a patient infection notification may be output based on the updated settings. In step 314, a behavior recommendation notification may be output based on the updated settings.

### Machine Learning Model

Described herein are systems, devices, and methods for constructing a machine learning model that processes and detects patient infection. The machine learning model can be used, for example, to determine a measure of infection (e.g., an estimate of probability of infection), as described above with reference to FIG. 2. In some embodiments, a machine learning model may be trained to output an optimal decision for a group of patients, which may be tuned to a specific patient by comparing which group of patients they are most similar to, using the model parameters optimized for (e.g., calibrated to) that group, and then tuning the types of errors allowable to the specific person based on their preferences. For example, a task-based machine learning algorithm can be trained or optimized to a group of individuals sharing a similar characteristic, e.g., pollen allergies, and used to determine a measure of infection for individuals having that characteristic.

In some embodiments, let x be the input vector of all of the data. The output may be the decision as to whether the input data vector is from class ω₁ indicating a “viral infection” or class ω₂ indicating a normal healthy state of a patient. Before we can make a good decision, we need to train a model to represent the probability of x given each of ω₁ and ω₂, or train up a model having a decision boundary that optimally separates data from classes ω₁ and ω₂.

Note that the probabilities of these two classes can vary for each patient: The model may take into account that the patient's geolocation, e.g. P(ω₁) is higher if a GPS geolocation corresponds to a viral hotspot. Similarly, P(ω₂) is higher for a patient having higher-than-average historic healthiness for not contracting a virus that is circulating. If the patient does not have historic data for this situation in their database, then the model defaults to using the population average based on demographic data.

Consideration may also be given to the cost of system errors. In some embodiments, the cost λ₁₂ may be the cost of a false positive, which may be a subjective patient metric. For example, one patient who doesn't like interruptions may associate a higher cost to being alerted to a viral infection if the system is wrong, while another patient, who has a high risk of serious complications, may associate a higher cost to not being notified if there actually is a viral infection, e.g., the cost λ₂₁.

Depending on the price of a person's healthcare and other preferences, a patient may consider the cost that the model is correct: λ₁₁, corresponds to correctly detected patient infection (no error, but λ₁₁>0) and the patient may choose to set λ₂₂=0 as usually there is no significant cost to staying healthy. In this case, it is assumed that the cost of being incorrect is greater than being correct such that λ₁₂>λ₂₂ and λ₂₁>λ₁₁. The model chooses to say that you have a viral infection (x is from ω₁) based on eqn. (1):

$$if \frac{P ( x \vert {ω}_{1} )}{P ( x \vert {ω}_{2} )} > \frac{{λ}_{12} - {λ}_{22}}{{λ}_{21} - {λ}_{11}} \cdot \frac{P ( {ω}_{2} )}{P ( {ω}_{1} )} \qquad eqn . ( 1 )$$

Otherwise, the model does not detect infection and notification is not output. In some embodiments, a viral infection probability may be estimated with a confidence score.

In some embodiments, a supervised learning model may be used to classify physiological data as healthy or infected. For example, a machine learning model may generate a feature space based on calibration data including healthy data and infected data. A function may be defined that represents a boundary in the feature space between the normal data and infected data. This function may also be further refined using unlabeled data or semi-supervised machine learning. Raw data may be input to the machine learning model and mapped into the feature space. Raw data may also be missing samples (e.g., from a faulty sensor or from data that contains motion artifacts), which may in some embodiments be interpolated or extrapolated using various statistical or machine learning techniques such as principle components analysis, multi-modal auto-encoders, or other means of using portions of good data to fill in parts that are missing. Missing data itself may also be labeled in ways that contribute to the decision being made. For example, certain kinds of missing data may have higher probability of occurring when a person is sick versus healthy, and these may also be used to influence the output of the algorithm. An output of the machine learning model may include classification of the raw data (and its interpolations) between normal data and infected data using the function developed by the model. This may improve the accuracy of patient infection detection.

In embodiments described herein, various machine learning and statistical modeling algorithms may be implemented, including classification algorithms, regression algorithms, neural network algorithms, supervised learning models, semi-supervised and unsupervised learning models, decision trees, random forests, mixture and multi-tasking methods, mixed-effects models, and the like. By applying a machine learning algorithm to calibration data, relationships may be developed and embodied in the model. Furthermore, the calibrated machine learning model may be tested and iterated upon by using data of the same type as the calibration data. Accordingly, various data parameters may be analyzed (e.g., with a calibrated model as described herein) to generate one or more outputs used to detect patient infection. It should be understood that it is possible to calibrate a machine learning model using any suitable calibration data for any suitable data parameter.

One or more of the machine learning models may be configured to generate a feature space including a first set of data points associated with the calibration data. A function may be defined that represents a boundary in the feature space between data points from the first set of data points associated with the normal calibration data and data points from the first set of data points associated with the infected calibration data. A feature space may be generated including a first set of data points associated with the training data. A portion of the raw data may be identified as infected based at least in part on the subset of data points identified using the function. For example, data points from calibration data may be plotted on a feature space with a boundary (e.g., plane defined by a function) separating the normal data points from the infected data points. In some embodiments, a function may be fit to the data based on features associated with the normal calibration data and the infected calibration data. The function may be linear or non-linear. In some embodiments, a non-linear function may provide a better fit to the data for different types of infections and patient conditions.

The accuracy of the function may be validated by inputting a set of training/testing data not used in generating the function. Once the validity of the function has been satisfied, raw data may be mapped into the feature space and their geolocation relative to the boundary (e.g., plane) may be used to classify the raw data points as normal or infected.

In some embodiments, regression analysis may be utilized to determine the relation of monitored parameters and patient infection. Regression analysis may be performed between patient infections and measured patient data to generate patient or population-based thresholds for generating patient infection notifications. This analysis may be retrospective statistical regression analysis from one or a plurality of patient data. For example, non-linear logistic regressions may be performed. Once regressions are established, when a measured patient data indicates high probability of infection, patient infection notification may be output.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

### III. EXAMPLES

As described herein, patient infection may be derived from measured physiological data. Examples 1 and 2 below show plots of patient infection data from two example studies: Example Studies A and B. The dataset from Example Study A includes data for 39 patients enrolled in an 11 day-long protocol at London Imperial College. Patients were asked to stay isolated in a dedicated facility during the entirety of the trial. During the second day, each patient was inoculated with Influenza A-H1N1 virus. Symptoms and results of nasal swab were collected daily, together with 24 hour measured physiological data using the Empatica® E4 device that measures and communicates data from blood volume pulse via photoplethysmography, 3-axis accelerometer, electrodermal activity, and peripheral temperature. Using these signals, the device can derive heart rate, heart-rate variability, respiration, sleep/wake, NREM/REM staging, and physiological changes likely generated by the sympathetic nervous system.

The dataset from Example Study B includes data for 21 patients enrolled in a 9 day-long protocol at Virginia University. The movement of the patients were not restricted such that they were not isolated. During the fifth day, each patient was inoculated with Influenza A-H1N1 virus. Symptoms and results of nasal swab were collected daily, together with 24 hour measured physiological data using the E4 device.

### Example 1

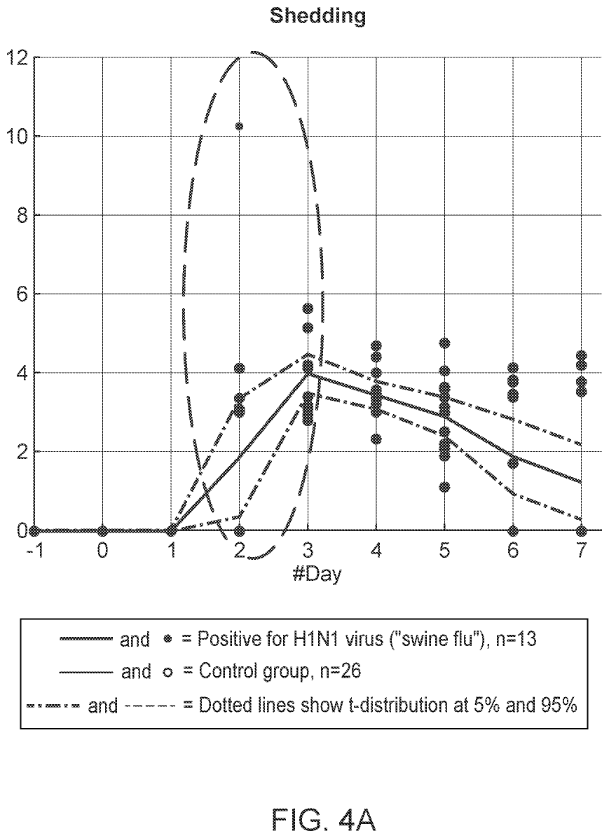

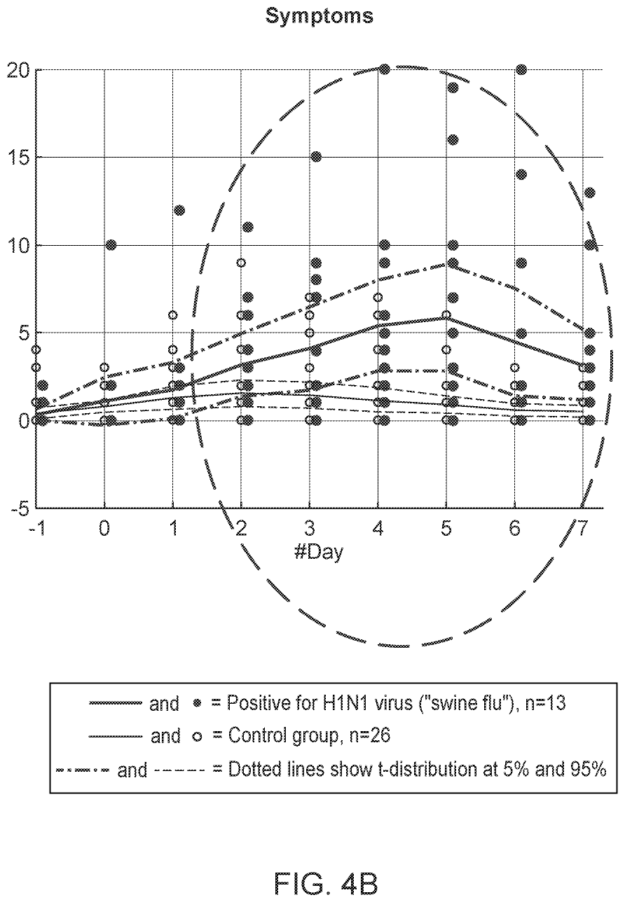

FIGS. 4A-4B are plots of patient infection data from Example Study A including an infected group and a control group. The FIG. 4A is a plot of viral shedding over time. FIG. 4B is a plot of the number of self-reported symptoms (e.g., aches and pains, coughing, sneezing, fever, difficulty breathing, rash, fatigue, etc.) over time. The highlighted portion of the plots corresponds to steady (two days or more) significant difference between the two groups. Symptoms are significantly different on days 2-7 (e.g., blue solid line above red dotted line at 95%).

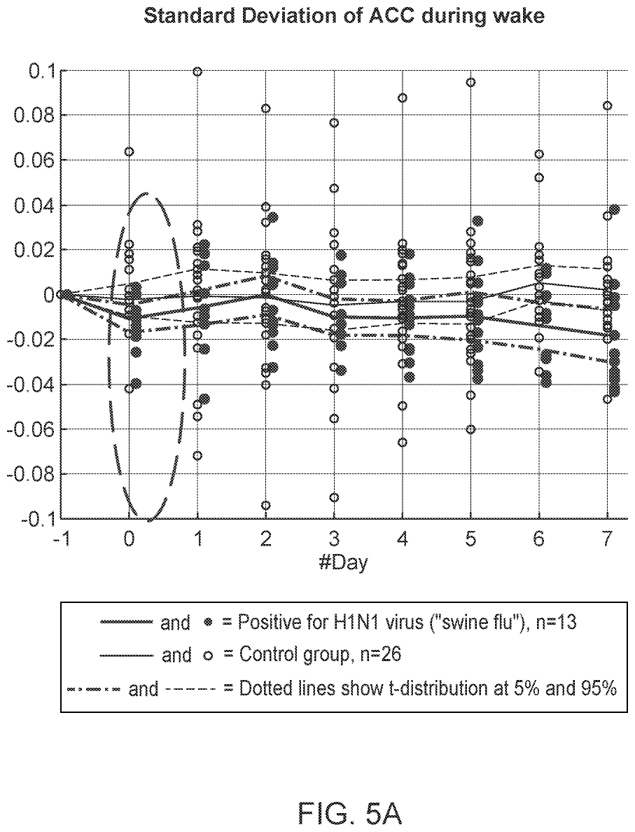

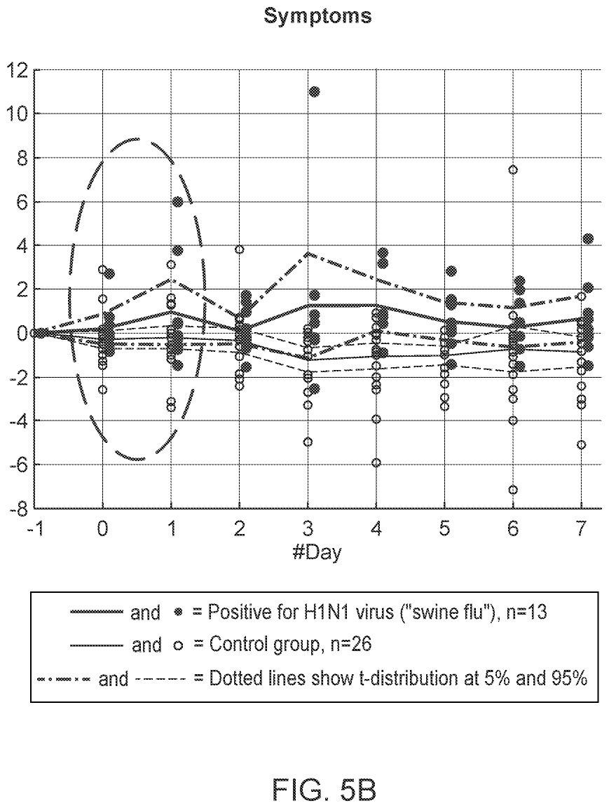

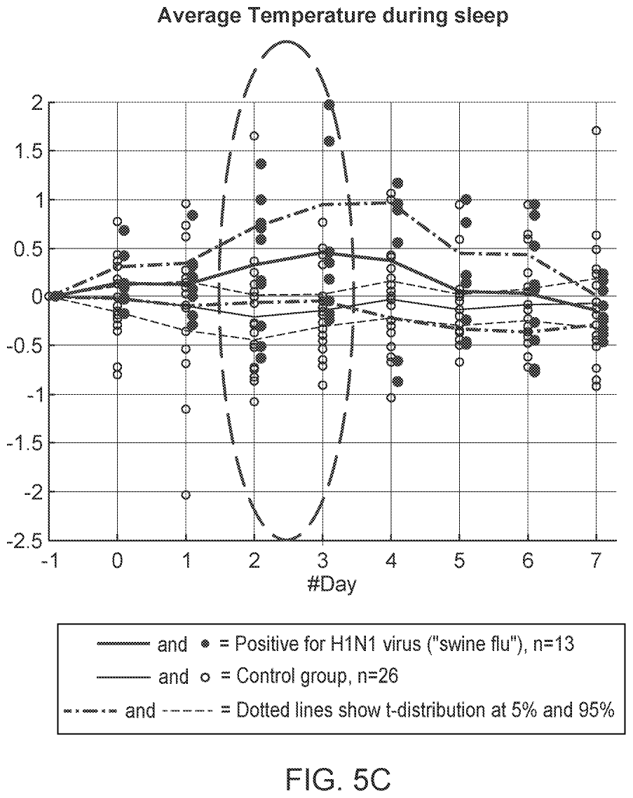

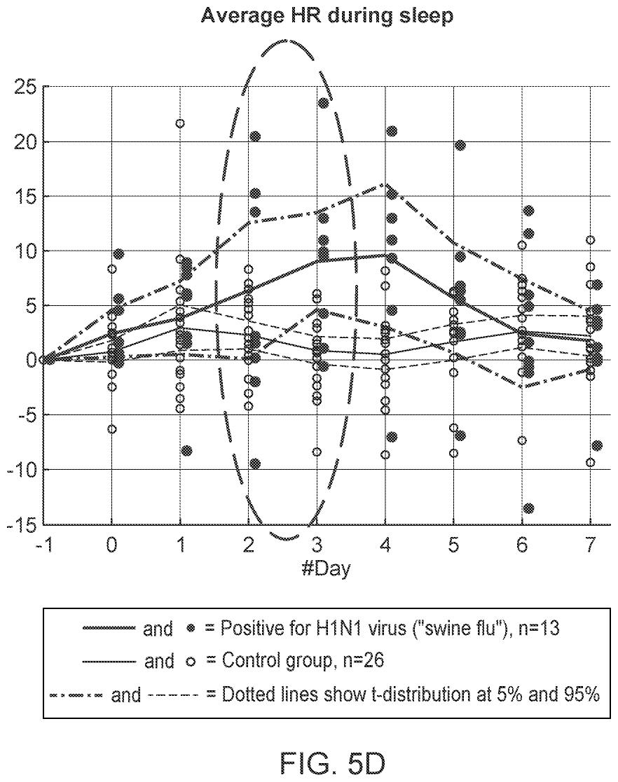

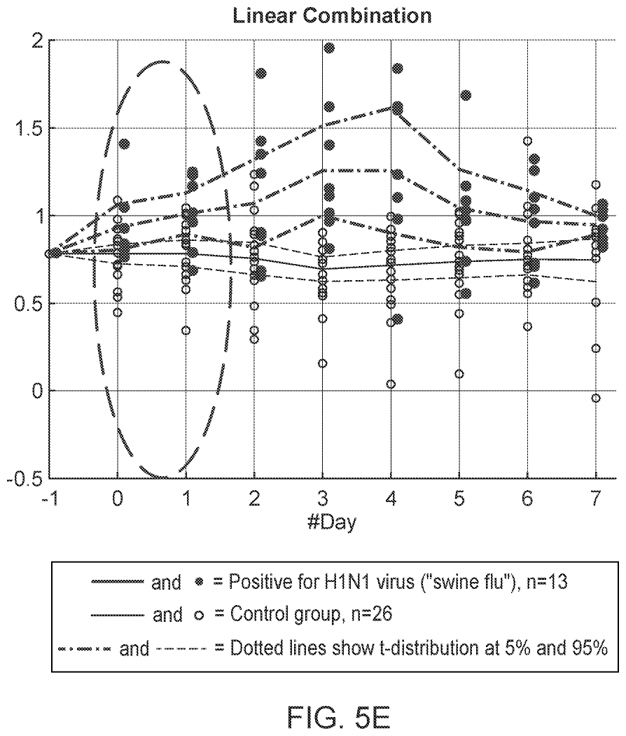

FIGS. 5A-5E are plots of measured physiological data from Example Study A. A reference baseline (e.g., non-infected state) is established on day “−1” and virus inoculation begins on day 0. In FIGS. 5A-5D, the y-axis value corresponds to the difference from day “−1”. FIG. 5A is a plot of acceleration (g) over time. FIG. 5B is a plot of skin conductance (μS) over time. FIG. 5C is a plot of temperature (Celsius) over time. FIG. 5D is a plot of heart rate (beats per minute) over time. The physiological signals shown are plotted relative to their day “−1” references.

FIG. 5E is a linear combination of the plots depicted in FIGS. 5A-5D. In particular, the linear combination is a sum of weighted components associated with inflammation.

As demonstrated by Example Study A, patients exhibiting viral shedding had physiological data (FIGS. 5A-5E) that deviated significantly from their baseline values days (e.g., two days or more) earlier than symptoms (FIG. 4B). Accordingly, systems, devices, and methods described herein are capable of detecting infection sooner than a patient may be experiencing symptoms of an infection. Stated differently, systems, devices, and methods described herein can be capable of forecasting a viral infection or the onset of symptoms associated with a viral infection or with the immune response activation triggered by a likely viral infection or when a vaccine generates an immune response.

### Example 2

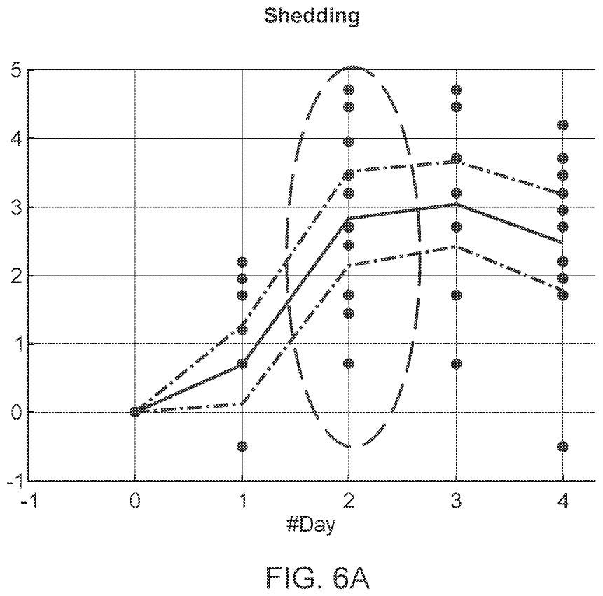

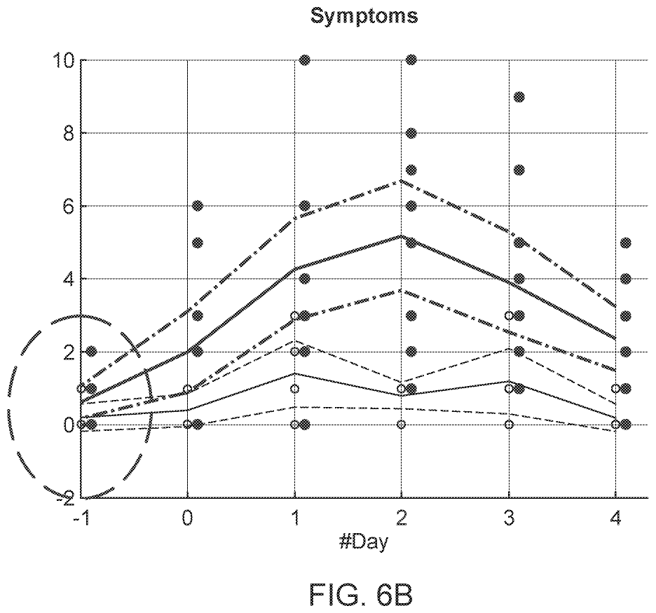

FIGS. 6A-6B are plots of physiological data from Example Study B. FIG. 6A is a plot of viral shedding over time. FIG. 6B is a plot of the number of self-reported symptoms (e.g., aches and pains, coughing, sneezing, fever, difficulty breathing, rash, fatigue, etc.) over time.

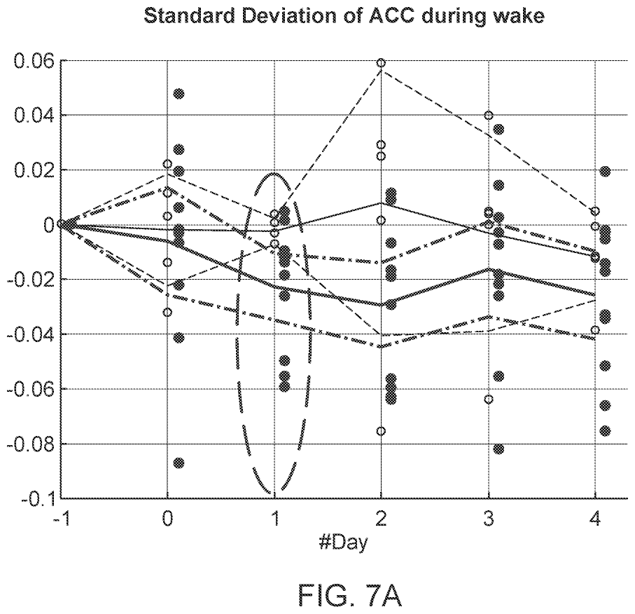

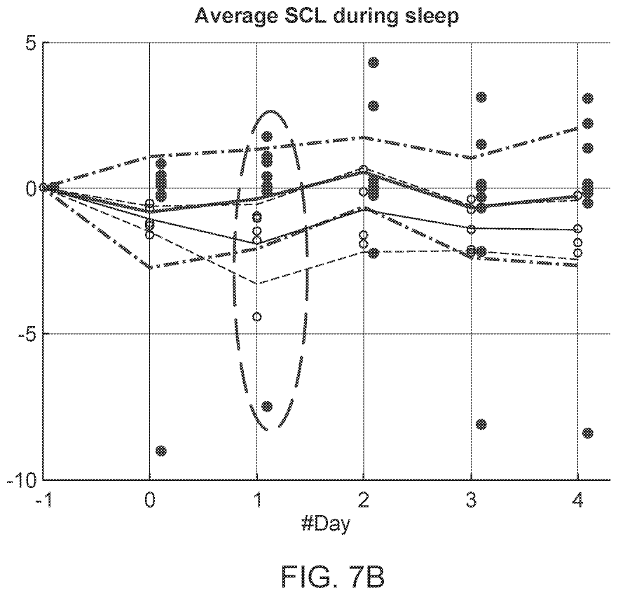

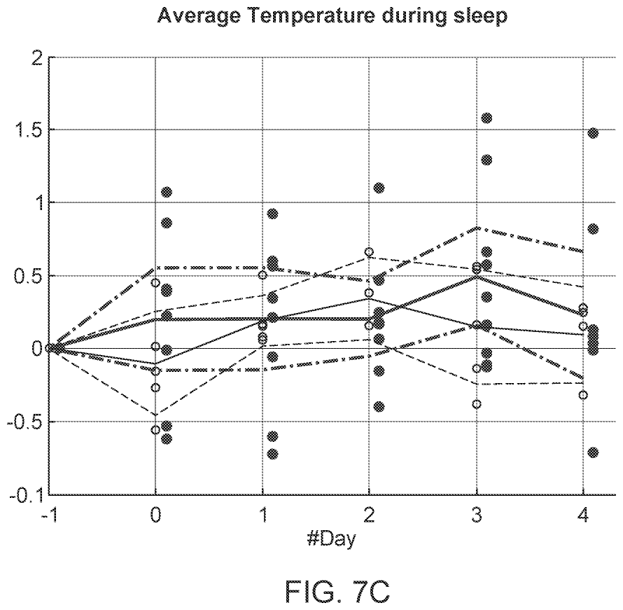

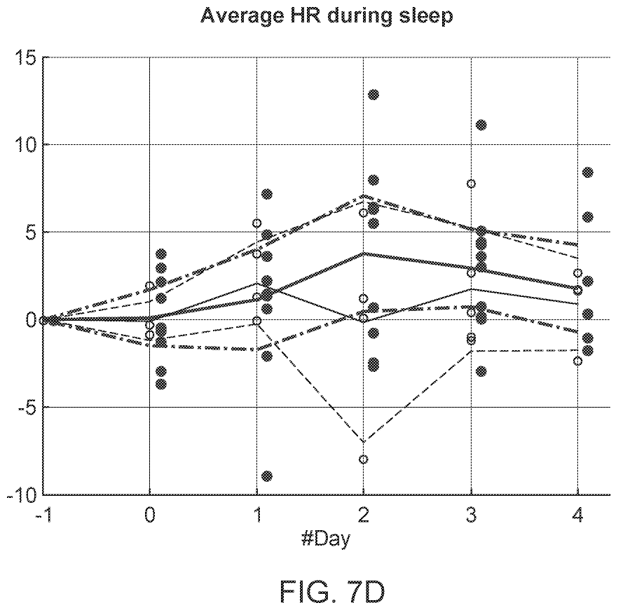

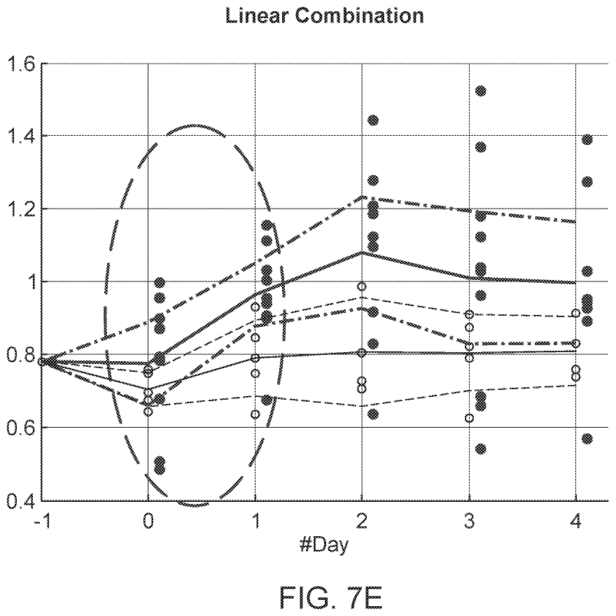

FIGS. 7A-7E are plots of physiological data from Example Study B. A reference baseline (e.g., non-infected state) is established on day “−1” and virus inoculation begins on day 0. In FIGS. 7A-7D, the y-axis value corresponds to the difference from day “−1”. FIG. 7A is a plot of acceleration (g) over time. FIG. 6B is a plot of skin conductance (μS) over time. FIG. 7C is a plot of temperature (Celsius) over time. FIG. 7D is a plot of heart rate (beats per minute) over time.

FIG. 7E is a linear combination of the plots depicted in FIGS. 6A-6D. In particular, the linear combination is a sum of weighted components associated with inflammation.

As demonstrated by Example Study B, patients exhibiting viral shedding had combined physiological data (FIG. 7E) that deviated significantly from their baseline values days (e.g., two days or more) earlier than symptoms (FIG. 6B). Accordingly, systems, devices, and methods described herein are capable of detecting infection sooner than a patient may be experiencing symptoms of an infection.

### Example 3

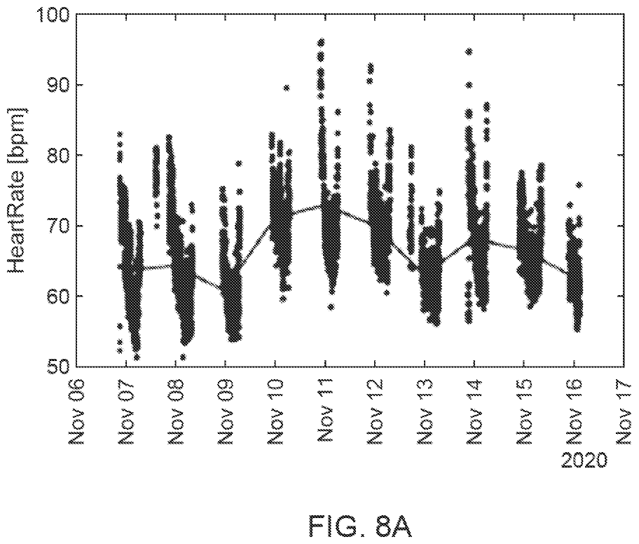

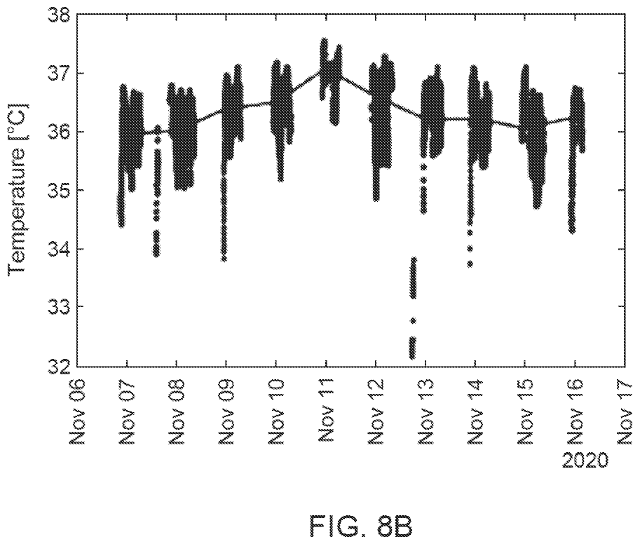

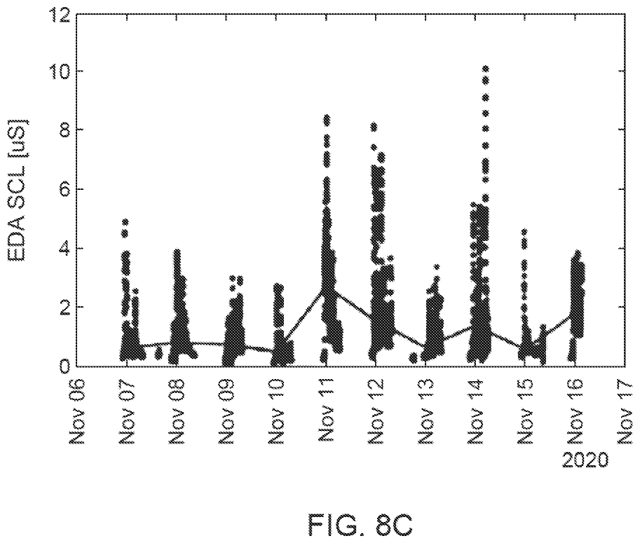

FIGS. 8A-8C are plots of physiological data of a patient infected with SARS-CoV-2. FIG. 8A is a plot of heart rate over time. FIG. 8B is a plot of temperature over time. FIG. 8C is a plot of electrodermal activity. Patient infection is known on day 1 (e.g., November 6). For each of the heart rate, temperature, and electrodermal activity. At about day 4 (e.g., November 10), an observable increase is apparent in each of the heart rate, temperature, and electrodermal activity plots that may be noticed by the patient. Accordingly, there may be a lag between patient infection and patient symptoms. This lag may differ based on the type of infection (e.g., COVID-19, influenza).

The specific examples and descriptions herein are exemplary in nature and embodiments may be developed by those skilled in the art based on the material taught herein without departing from the scope of the present invention, which is limited only by the attached claims.

1. A method, comprising:
receiving, from one or more sensors, physiological data of a patient measured by the one or more sensors, the physiological data including movement data associated with the patient;
identifying a resting time period during which the patient is at rest based on the movement data;
extracting, from the physiological data, data of a set of physiological parameters during a portion of the resting time period;
determining a derived measure for each physiological parameter from the set of physiological parameters based on the data of the set of physiological parameters extracted during the portion of the resting time period;
adjusting the derived measure for each physiological parameter from the set of physiological parameters based on a baseline measure for the corresponding physiological parameter from the set of physiological parameters to produce a set of adjusted measures, the baseline measure associated with physiological data of the patient collected during a time period prior to the resting time period;
inputting the set of adjusted measures into a model for predicting an infection risk of the patient to obtain a predictive value indicative of an infection level of the patient; and
determining an infection state of the patient based on the predictive value.

2. The method of claim 1, wherein the derived measure for each physiological parameter from the set of physiological parameters is an aggregate value of the data extracted for that physiological parameter during the portion of the resting time period.

3. The method of claim 2, wherein the aggregate value is based on one or more of: a mean, a median, a standard deviation, a variance, or a higher order statistic.

4. The method of claim 1, wherein the set of physiological parameters includes two or more of: heart rate, heart rate variability, skin temperature, respiration rate, skin conductance, skin resistance, skin potential, motion, blood-oxygen level, protein, or cytokines.

5. The method of claim 1, wherein the adjusting the derived measure for each physiological parameter from the set of physiological parameters includes determining a change between the derived measure and the baseline measure for each physiological parameter from the set of physiological parameters.

6. The method of claim 1, further comprising:
receiving at least one of demographic data or medical data of the patient; and
adjusting the data of the set of physiological parameters extracted during the portion of the resting time period based on the at least one of the demographic data or the medical data,
the determining the derived measure for each physiological parameter from the set of physiological parameters being after the normalizing the rest data.

7. The method of claim 6, further comprising adjusting the model for predicting an infection risk of the patient based on at least one of the demographic data or the medical data.

8. The method of claim 1, further comprising:
normalizing the data of the set of physiological parameters extracted during the portion of the resting time period.

9. The method of claim 8, wherein determining the derived measure for each physiological parameter from the set of physiological parameters is after the normalizing the rest data.

10. The method of claim 8, wherein normalizing the data is based on minimum-maximum normalization.

11. The method of claim 1, wherein determining the infection state of the patient includes:
determining whether the predictive value is greater than a predefined threshold value; and
in response to the predictive value being greater than the predefined threshold value, determining that the patient is infected.

12. The method of claim 1, wherein the predefined threshold value is adjustable.

13. The method of claim 1, wherein the model is calibrated using training data including physiological data associated with a set of users, the set of users and the patient having a common set of characteristics.

14. The method of claim 1, wherein the model is configured to apply a set of weights to the set of adjusted measures, the set of weights being calibrated using training data including physiological data associated with a set of users.

15. The method of claim 14, wherein the physiological data of the set of users is associated with one or more of a geolocation and weather.

16. The method of claim 1, wherein the model defines a non-linear function.

17. The method of claim 1, wherein the time period prior to the resting time period is at least about 24 hours prior to the resting time period.

18. The method of claim 1, wherein time period prior to the resting time period is a predefined period of time prior to the resting time period, the predefined period of time based on a type of infection.

19. The method of claim 1, further comprising monitoring the infection state of the patient over time to identify one or more of a change in the infection state of the patient, an infection resolution, and estimating a duration of an infection.

20. The method of claim 1, wherein the time period and the resting time period are based on a periodic time interval.

21. The method of claim 20, wherein the periodic time interval comprises one or more of a calendar cycle, hormonal cycle, lunar cycle, circadian rhythm, and multidien rhythm, and work schedule.

22. The method of claim 1, wherein adjusting the derived measure is based on a weather associated with the patient.

23. The method of claim 1, further comprising determining an infection risk based at least in part on a geolocation of the patient.

24. An apparatus, comprising:
a memory; and
a processor operatively coupled to the memory and a set of sensors, the processor configured to execute instructions stored in the memory to:
receive, from the set of sensors, physiological data of a patient measured by the set of sensors;
extract, from the physiological data, data of a set of physiological parameters during a resting time period associated with the patient;
determine a derived measure for each physiological parameter from the set of physiological parameters based on the data of the set of physiological parameters extracted during the resting time period;
adjust the derived measure for each physiological parameter from the set of physiological parameters based on a baseline measure for the corresponding physiological parameter from the set of physiological parameters to produce a set of adjusted measures, the baseline measure associated with physiological data of the patient collected during a time period prior to the resting time period;
determine a predictive value indicative of an infection level of the patient using a model for predicting an infection risk of the patient and the set of adjusted measures; and
determine an infection state of the patient based on the predictive value.

25. The apparatus of claim 24, further comprising:
a wearable device including the set of sensors.

26. The apparatus of claim 24, wherein the derived measure for each physiological parameter from the set of physiological parameters is an aggregate value of the data extracted for that physiological parameter during the portion of the resting time period.

27. The apparatus of claim 24, wherein the set of physiological parameters includes two or more of: heart rate, heart rate variability, skin temperature, respiration rate, skin conductance, blood-oxygen levels, skin resistance, skin potential, motion, or cytokines.

28. The apparatus of claim 24, wherein the processor is configured to execute the instruction to adjust the derived measure for each physiological parameter from the set of physiological parameters by determining a change between the derived measure and the baseline measure for each physiological parameter from the set of physiological parameters.

29. The apparatus of claim 24, wherein the model is configured to apply a set of weights to the set of adjusted measures, the set of weights being calibrated using training data including physiological data associated with a set of users.

30. The apparatus of claim 24, wherein the model defines a non-linear function.
